(19) United States
(12) Patent Application Publication
(10) Pub. No.: US 2025/0322926 A1
(43) Pub. Date: Oct. 16, 2025

(54) EARLY ONSET RESPONSE, FAVORABLE TOLERABILITY, AND SIDE EFFECT PROFILE IN THE TREATMENT OF FIBROMYALGIA

(71) Applicant: Tonix Pharmaceuticals Holding Corp., Chatham, NJ (US)

(72) Inventors: Seth Lederman, Austin, TX (US); Gregory M. Sullivan, Barryville, NY (US)

(21) Appl. No.: 18/988,194

(22) Filed: Dec. 19, 2024

Related U.S. Application Data
(60) Provisional application No. 63/612,352, filed on Dec. 19, 2023.
(60) Provisional application No. 63/618,892, filed on Jan. 8, 2024.

(51) Int. Cl. G16H 20/10; G16H 10/20; G16H 70/40

(52) U.S. Cl. CPC G16H 20/10; G16H 10/20; G16H 70/40

(57) ABSTRACT

The present disclosure provides methods for treating or managing fibromyalgia and its associated symptoms in a subject in need hereof characterized by an early onset of one or more of: (1) a reduction in widespread pain characterized by about 0.3 or more difference in the LS mean change in the NRS Pain Score; (2) a reduction in sleep disturbance characterized by about 2.9 or more difference in the LS Mean change in PROMIS Sleep Disturbance T-score; (3) a reduction in fatigue characterized by about 2.0 or more difference in the LS Mean change in PROMIS Fatigue T-score; or (4) an improved sleep quality characterized by about 0.5 or more difference in the LS Mean change in NRS Sleep Quality Score, each as compared to placebo, comprising administering to a subject in need thereof 2.8 mg or 5.6 mg cyclobenzaprine HCl once daily in one or more dosage units by transmucosal administration. The present disclosure also provides methods for preventing or avoiding one or more of a clinically meaningful change in mean weight, a clinically meaningful change in mean systolic blood pressure or mean diastolic blood pressure, or a decline in sexual functioning in conjunction with treating or managing fibromyalgia or its associated symptoms in a subject in need thereof, the treatment or management being characterized by the transmucosal administration of cyclobenzaprine HCl in one or more dosage units (e.g., a first dosage unit or a second dosage unit). The cyclobenzaprine HCl of these methods is in the form of a mannitol eutectic (e.g., a 75%±2% by weight cyclobenzaprine HCl and 25%±2% by weight β-mannitol eutectic) and the one or more dosage units further comprise a basifying agent.

### CROSS-REFERENCE TO RELATED APPLICATIONS

This application claims priority to and benefit of United States Provisional Aplication No. 63/612,352, filed Dec. 19, 2023, and U.S. Provisional Application No. 63/618,892, filed Jan. 8, 2024, the contents of each are hereby incorporated by reference in their entireties.

### BACKGROUND

Cyclobenzaprine, or 3-(5H-dibenzola[a,d]cyclohepten-5-ylidene)-N,N-dimethyl-1 propanamine, was first approved by the U.S. Food and Drug Administration in 1977 for the treatment of acute muscle spasms of local origin. (Katz and Dube, 1988).

An estimated 6-12 million adults in the United States have fibromyalgia and 90% of whom are women. Fibromyalgia, or fibromyalgia syndrome, is considered a central nervous system disorder with symptoms including chronic widespread pain, nonrestorative sleep, fatigue, diminished cognition and mood disturbances. These symptoms are believed to result from inappropriate pain signaling in the central nervous system in the absence of peripheral injury. Fibromyalgia causes significant impairment in all areas in life where patients present with lower levels of health-related quality of life, such as reduced daily functioning and interference with work (e.g., loss of productivity and disability). There is an unmet need to treat or manage fibromyalgia as fewer than half of the fibromyalgia patients can receive complete relief from the current three FDA-approved drugs. Moreover, the three FDA approved drugs also have tolerability and side effect issues, particularly in the areas exhibiting clinically meaningful adverse effects on weight, blood pressure and sexual functioning, that limit their use in the treatment and management of fibromyalgia and its associated symptoms. There is also substantial off-label use of narcotic painkillers and prescription sleep aids. Among those diagnosed, more than one-third have used prescription opioids as a means of treatment. Cyclobenzaprine HCl, as described in various embodiments of this disclosure, meets this unmet need and improves pain, sleep quality and fatigue for subjects suffering from fibromyalgia.

By contrast, the Cyclobenzaprine HCl treatment, as described in various embodiments of this disclosure, surprisingly has favorable tolerability and side effect profiles, particularly in the context of avoiding clinically meaning effects on weight, blood pressure, and sexual functioning when used to treat or manage fibromyalgia in subject in need thereof.

### SUMMARY OF THIS DISCLOSURE

Some embodiments of this disclosure are:
1. A method for treating or managing fibromyalgia and its associated symptoms in a subject in need thereof characterized by an early onset of one or more of: (1) a reduction in widespread pain characterized by about 0.3 or more difference in the least squares (LS) mean change in the Numerical Rating Scale (NRS) Pain Score; (2) a reduction in sleep disturbance characterized by about 2.9 or more difference in the LS Mean change in PROMIS Sleep Disturbance T-score; (3) a reduction in fatigue characterized by about 2.0 or more difference in the LS Mean change in PROMIS Fatigue T-score; or (4) an improved sleep quality characterized by about 0.5 or more difference in the LS Mean change in NRS Sleep Quality Score, each as compared to placebo, comprising administering to a subject in need thereof 5.6 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.
2. A method for treating or managing fibromyalgia and its associated symptoms in a subject in need thereof characterized by an early onset of one or more of: (1) a reduction in widespread pain characterized by about 0.3 or more difference in the least squares (LS) mean change in the Numerical Rating Scale (NRS) Pain Score; (2) a reduction in sleep disturbance characterized by about 2.9 or more difference in the LS Mean change in PROMIS Sleep Disturbance T-score; (3) a reduction in fatigue characterized by about 2.0 or more difference in the LS Mean change in PROMIS Fatigue T-score; or (4) an improved sleep quality characterized by about 0.5 or more difference in the LS Mean change in NRS Sleep Quality Score, each as compared to placebo, comprising administering to a subject in need thereof 2.8 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.
3. The method for treating or managing according to embodiment 1 or 2, wherein the cyclobenzaprine HCl-mannitol eutectic is a 75%±2% by weight cyclobenzaprine HCl and 25%±2% by weight β-mannitol eutectic.
4. The method for treating or managing according to embodiment 1, wherein the one or more dosage units comprising the cyclobenzaprine HCl-mannitol eutectic and the basifying agent are two dosage units, each dosage unit comprising 2.8 mg of cyclobenzaprine HCl.
5. The method for treating or managing according to embodiment 2, wherein the one or more dosage units comprising the cyclobenzaprine HCl-mannitol eutectic and the basifying agent is one dosage unit, the dosage unit comprising 2.8 mg of cyclobenzaprine HCl.
6. A multiple-variable dose method for treating or managing fibromyalgia and its associated symptoms in a subject in need thereof characterized by an early onset of one or more of: (1) a reduction in widespread pain characterized by about 0.3 or more difference in the least square (LS) mean change in the Numerical Rating Scale (NRS) Pain Score; (2) a reduction in sleep disturbance characterized by about 2.9 or more difference in the LS Mean change in PROMIS Sleep Disturbance T-score; (3) a reduction in fatigue characterized by about 2.0 or more difference in the LS Mean change in PROMIS Fatigue T-score; or (4) an improved sleep quality characterized by about 0.5 or more difference in the LS Mean change in NRS Sleep Quality Score, each as compared to placebo, comprising the transmucosal administration of:
   one or more of a first dosage unit comprising in total 2.8 mg of cyclobenzaprine HCl to the subject once daily for about two weeks; and
   one or more of a subsequent second dosage unit comprising in total 5.6 mg of cyclobenzaprine HCl to the subject once daily for as long as needed,
wherein the cyclobenzaprine HCl is in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and wherein the first dosage unit and the second dosage unit further comprise a basifying agent.
7. The method for treating or managing according to embodiment 6, wherein the cyclobenzaprine HCl-mannitol eutectic that characterizes said first dosage unit and said second dosage units is a 75%±2% by weight cyclobenzaprine HCl and 25%±2% by weight β-mannitol eutectic.
8. The method for treating or managing according to any one of embodiments 1-7, wherein the one or more dosage units comprising the cyclobenzaprine HCl-mannitol eutectic and basifying agent are administered at bedtime.
9. The method for treating or managing according to any one of embodiments 1-8, wherein said transmucosal administration comprises sublingual, buccal, intranasal or palatal administration.
10. The method for treating or managing according to embodiment 9, wherein said transmucosal administration is sublingual administration.
11. The method for treating or managing according to embodiment 10, wherein the dosage form is a sublingual tablet, a sublingual film, a sublingual liquid, sublingual powder, or a sublingual spray solution.
12. The method for treating or managing according to any one of embodiments 1-11, wherein the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate, trisodium citrate, borate, hydroxide, silicate, nitrate, dissolved ammonia, bicarbonate, and sulfide.
13. The method for treating or managing according to embodiment 12, wherein the basifying agent is dipotassium hydrogen phosphate.
14. The method for treating or managing according to any one of embodiments 1-13, wherein the pain is measured by a daily diary pain severity score using a numerical rating scale.
15. The method for treating or managing according to any one of embodiments 1-14, wherein the early onset of the reduction in widespread pain begins about 1-4 weeks after administration.
16. The method for treating or managing according to any one of embodiments 1-15, wherein the early onset of the reduction in widespread pain begins about 1-3 weeks after administration.
17. The method for treating or managing according to any one of embodiments 1-16, wherein the early onset of the reduction in widespread pain begins about 1-2 weeks after administration.
18. The method for treating or managing according to any one of embodiments 1-17, wherein the early onset of the reduction in widespread pain begins about 1 week after administration.
19. The method for treating or managing according to any one of embodiments 1-17, wherein the early onset of the reduction in widespread pain begins about 2 weeks after administration.
20. The method for treating or managing according to any one of embodiments 1-16, wherein the early onset of the reduction in widespread pain begins about 3 weeks after administration.
21. The method for treating or managing according to any one of embodiments 1-15, wherein the early onset of the reduction in widespread pain begins about 4 weeks after administration.
22. The method for treating or managing according to any one of embodiments 1-21, wherein the early onset of the reduction in sleep disturbance begins about 1-6 weeks after administration.
23. The method for treating or managing according to any one of embodiments 1-22, wherein the early onset of the reduction in sleep disturbance begins about 1-4 weeks after administration.
24. The method for treating or managing according to any one of embodiments 1-23, wherein the early onset of the reduction in sleep disturbance begins about 1-2 weeks after administration.
25. The method for treating or managing according to any one of embodiments 1-24, wherein the early onset of the reduction in sleep disturbance begins about 2 weeks after administration.
26. The method for treating or managing according to any one of embodiments 1-23, wherein the early onset of the reduction in sleep disturbance begins about 4 weeks after administration.
27. The method for treating or managing according to any one of embodiments 1-22, wherein the early onset of the reduction in sleep disturbance begins about 6 weeks after administration.
28. The method for treating or managing according to any one of embodiments 1-27, wherein the early onset of the reduction in fatigue begins about 1-6 weeks after administration.
29. The method for treating or managing according to any one of embodiments 1-28, wherein the early onset of the reduction in fatigue begins about 1-4 weeks after administration.
30. The method for treating or managing according to any one of embodiments 1-29, wherein the early onset of the reduction in fatigue begins about 1-2 weeks after administration.
31. The method for treating or managing according to any one of embodiments 1-30, wherein the early onset of the reduction in fatigue begins about 2 weeks after administration.
32. The method for treating or managing according to any one of embodiments 1-29, wherein the early onset of the reduction in fatigue begins about 4 weeks after administration.
33. The method for treating or managing according to any one of embodiments 1-28, wherein the early onset of the reduction in fatigue begins about 6 weeks after administration.
34. The method for treating or managing according to any one of embodiments 1-33, wherein the sleep quality is prior night sleep quality.
35. The method for treating or managing according to any one of embodiments 1-34, wherein the early onset of the improved sleep quality begins about 1-4 weeks after administration.
36. The method for treating or managing according to any one of embodiments 1-35, wherein the early onset of the improved sleep quality begins about 1-3 weeks after administration.
37. The method for treating or managing according to any one of embodiments 1-36, wherein the early onset of the improved sleep quality begins about 1-2 weeks after administration.
38. The method for treating or managing according to any one of embodiments 1-37, wherein the early onset of the improved sleep quality begins about 1 week after administration.
39. The method for treating or managing according to any one of embodiments 1-37, wherein the early onset of the improved sleep quality begins about 2 weeks after administration.
40. The method for treating or managing according to any one of embodiments 1-36, wherein the early onset of the improved sleep quality begins about 3 weeks after administration.
41. The method for treating or managing according to any one of embodiments 1-35, wherein the early onset of the improved sleep quality begins about 4 weeks after administration.
42. The method for treating or managing according to any one of embodiments 1-41, wherein the subject is human.
43. A method for preventing or avoiding one or more of a clinically meaningful change in mean weight, a clinically meaningful change in mean systolic blood pressure or mean diastolic blood pressure, or a decline in sexual functioning in conjunction with treating or managing fibromyalgia or its associated symptoms in a subject in need thereof, the treatment or management being characterized by the transmucosal administration of in total 5.6 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65% =2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the one or more dosage units further comprise a basifying agent.
44. A method for preventing or avoiding one or more of a clinically meaningful change in mean weight, a clinically meaningful change in mean systolic blood pressure or mean diastolic blood pressure, or a decline in sexual functioning in conjunction with treating or managing fibromyalgia or its associated symptoms in a subject in need thereof, the treatment or management being characterized by the transmucosal administration of 2.8 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the one or more dosage units further comprise a basifying agent.
45. The method for preventing or avoiding according to embodiment 43 or 44, wherein the cyclobenzaprine HCl-mannitol eutectic is a 75%±2% by weight cyclobenzaprine HCl and 25%±2% by weight β-mannitol eutectic.
46. The method for preventing or avoiding according to embodiment 43, wherein the one or more dosage units comprising the cyclobenzaprine HCl-mannitol eutectic and the basifying agent are two dosage units, each dosage unit comprising 2.8 mg of cyclobenzaprine HCl.
47. The method for preventing or avoiding according to embodiment 44, wherein the one or more dosage units comprising the cyclobenzaprine HCl-mannitol eutectic and the basifying agent is one dosage unit, the dosage unit comprising 2.8 mg of cyclobenzaprine HCl.
48. A method for preventing or avoiding one or more of a clinically meaningful change in mean weight, a clinically meaningful change in mean systolic blood pressure or mean diastolic blood pressure, or a decline in sexual functioning in conjunction with treating or managing fibromyalgia or its associated symptoms in a subject in need thereof, the treatment or management being characterized by the transmucosal administration of:
   one or more of a first dosage unit comprising in total 2.8 mg of cyclobenzaprine HCl to the subject once daily for about two weeks; and
   one or more of a subsequent second dosage unit comprising in total 5.6 mg of cyclobenzaprine HCl to the subject once daily for as long as needed,
wherein the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the first dosage unit and the second dosage unit further comprise a basifying agent.
49. The method for preventing or avoiding according to embodiment 48, wherein the cyclobenzaprine HCl-mannitol eutectic that characterizes said first dosage unit and said second dosage unit is a 75%±2% by weight cyclobenzaprine HCl and 25%±2% by weight β-mannitol eutectic.
50. The method for preventing or avoiding according to any one of embodiments 43-49, wherein the one or more dosage units comprising the cyclobenzaprine HCl-mannitol eutectic and basifying agent are administered at bedtime.
51. The method for preventing or avoiding according to any one of embodiments 43-50, wherein said transmucosal administration comprises sublingual, buccal, intranasal or palatal administration.
52. The method for preventing or avoiding according to embodiment 51, wherein said transmucosal administration is sublingual administration.
53. The method for preventing or avoiding according to embodiment 52, wherein the dosage form is a sublingual tablet, a sublingual film, a sublingual liquid, sublingual powder, or a sublingual spray solution.
54. The method for preventing or avoiding according to any one of embodiments 43-53, wherein the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate, trisodium citrate, borate, hydroxide, silicate, nitrate, dissolved ammonia, bicarbonate, and sulfide.
55. The method for preventing or avoiding according to embodiment 54, wherein the basifying agent is dipotassium hydrogen phosphate.
56. The method for preventing or avoiding according to any one of embodiments 43-55, wherein the method prevents or avoids a clinically meaningful change in mean weight.
57. The method for preventing or avoiding according to embodiments 56, wherein the clinically meaningful change in mean weight comprises a clinically meaningful increase in mean weight.
58. The method for preventing or avoiding according to embodiments 56, wherein the clinically meaningful change in mean weight comprises a clinically meaningful decrease in mean weight.
59. The method for preventing or avoiding according to any one of embodiments 43-58, wherein the method prevents or avoids a clinically meaningful change in one or more of mean systolic blood pressure or mean diastolic blood pressure.
60. The method for preventing or avoiding according to embodiment 59, wherein the clinically meaningful change in one or more of the mean systolic blood pressure or the mean diastolic blood pressure comprises a clinically meaningful increase in one or more of the mean systolic blood pressure or the mean diastolic blood pressure.
61. The method for preventing or avoiding according to any one of embodiments 43-60, wherein the method prevents or avoids a decline in sexual functioning.
62. The method for preventing or avoiding according to any one of embodiments 43-61, wherein said sexual functioning comprises one or more of orgasm or completion, arousal or excitement, desire or interest, frequency of desire, or pleasure.
63. The method for preventing or avoiding according to any one of embodiments 43-47 and 50-62, wherein the subject is transmucosally administered one or more dosage units comprising in total 5.6 mg cyclobenzaprine HCl once daily for up to 14 weeks.
64. The method for preventing or avoiding according to any one of embodiments 43-47 and 50-62, wherein the subject is transmucosally administered one or more dosage units comprising in total 5.6 mg cyclobenzaprine HCl once daily for 14 weeks.
65. The method for preventing or avoiding according to any one of embodiments 43-47 and 50-62, wherein the subject is transmucosally administered one or more dosage units comprising in total 5.6 mg cyclobenzaprine HCl once daily for 14 or more weeks.
66. The method for preventing or avoiding according to any one of embodiments 48-62, wherein the subject is transmucosally administered one or more of the second dosage unit comprising in total 5.6 mg cyclobenzaprine HCl once daily for up to 12 weeks.
67. The method for preventing or avoiding according to any one of embodiments 48-62, wherein the subject is transmucosally administered one or more of the second dosage unit comprising in total 5.6 mg cyclobenzaprine HCl once daily for 12 weeks.
68. The method for preventing or avoiding according to any one of embodiments 48-62, wherein the subject is transmucosally administered one or more of the second dosage unit comprising in total 5.6 mg cyclobenzaprine HCl once daily for 12 or more weeks.
69. The method for treating or managing fibromyalgia or its associated symptoms according to any one of embodiments 43-68, wherein the subject is human.

### BRIEF DESCRIPTION OF DRAWINGS

FIG. 1 shows a graph of least squares (LS) mean (SE) change in Numerical Rating Scale (NRS) pain score (Primary Endpoint). Pain score was measured and compared between Placebo (N=225) and Cyclobenzaprine HCl (N=231) groups once a week for 14 weeks. SE refers to standard error. * p<0.01; ** p<0.001; and *** p<0.0001.

FIG. 2 shows a bar graph of percentage much improved or very much improved over time (Weeks 2, 6, 10, and 14) from the Patient Global Impression of Change Responder Analysis (PGIC). * p<0.01 and ** p<0.001. PGIC was measured and compared between Placebo (N=225) and Cyclobenzaprine HCl (N=231) groups.

FIG. 3 shows a graph of least squares (LS) mean (SE) change in Fibromyalgia Impact Questionnaire-Revised (FIQ-R) Symptoms Domain. FIQ-R Symptoms was measured and compared between Placebo (N=225) and Cyclobenzaprine HCl (N=231) groups. SE refers to standard error. * p=0.003 and *** p<0.0001.

FIG. 4 shows a graph of least squares (LS) mean (SE) change in Fibromyalgia Impact Questionnaire-Revised (FIQ-R) Function Domain. FIQ-R Function Domain was measured and compared between Placebo (N=225) and Cyclobenzaprine HCl (N=231) groups. SE refers to standard error. ** p<0.01.

FIG. 5 shows a graph of least squares (LS) mean (SE) change in Patient-Reported Outcomes Measurement Information System (PROMIS) Sleep Disturbance (SD) T-score over time (Weeks 0, 2, 6, 10, and 14). PROMIS SD was measured and compared between Placebo (N=225) and Cyclobenzaprine HCl (N=231) groups. SE refers to standard error. * p<0.001 and *** p<0.00001.

FIG. 6 shows a graph of least squares (LS) mean (SE) change in Patient-Reported Outcomes Measurement Information System (PROMIS) Fatigue T-Score over time (Weeks 0, 2, 6, 10, and 14). PROMIS Fatigue was measured and compared between Placebo (N=225) and Cyclobenzaprine HCl (N=231) groups. SE refers to standard error. * p<0.01 and *** p<0.0001.

FIG. 7 shows a graph of least squares (LS) mean (SE) change in Numerical Rating Scale (NRS) sleep quality score. Sleep quality score was measured and compared between Placebo (N=225) and Cyclobenzaprine HCl (N=231) groups once a week for 14 weeks. SE refers to standard error. * p<0.01; ** p<0.001; and *** p<0.0001.

FIG. 8 shows a continuous pain responder graph as measured by percent of subjects treated with placebo or Cyclobenzaprine HCl versus percentage reduction in pain from baseline to Week 14. * p=0.011 and ** p<0.001.

FIG. 9 shows a vertical box plot of systolic blood pressure (mmHg) measurements at baseline and at Weeks 2, 6, 10, and 14. Systolic blood pressure was measured and compared between Placebo (N=226) and Cyclobenzaprine HCl (N=231) groups. The solid horizontal line is the mean for Placebo and the dashed horizontal line is the mean for Cyclobenzaprine HCl. The boxes are the 25^{th} and 75^{th} percentiles and the vertical lines begin and end at the 5^{th} and 95^{th} percentiles. CBP HCl refers to Cyclobenzaprine HCl.

FIG. 10 shows a vertical box plot of diastolic blood pressure (mmHg) measurements at baseline and at Weeks 2, 6, 10, and 14. Diastolic blood pressure was measured and compared between Placebo (N=226) and Cyclobenzaprine HCl (N=231) groups. The solid horizontal line is the mean for Placebo and the dashed horizontal line is the mean for Cyclobenzaprine HCl. The boxes are the 25^{th} and 75^{th} percentiles and the vertical lines begin and end at the 5^{th} and 95^{th} percentiles. CBP HCl refers to Cyclobenzaprine HCl.

FIG. 11 shows a vertical box plot of weight (kg) measurements at baseline and at Weeks 2, 6, 10, and 14. Weight was measured and compared between Placebo (N=226) and Cyclobenzaprine HCl (N=231) groups. The solid horizontal line is the mean for Placebo and the dashed horizontal line is the mean for Cyclobenzaprine HCl. The boxes are the 25^{th} and 75^{th} percentiles and the vertical lines begin and end at the 5^{th} and 95^{th} percentiles. CBP HCl refers to Cyclobenzaprine HCl.

FIG. 12 shows a graph of least squares (LS) mean difference in score for the Changes in Sexual Functioning Questionnaire between Cyclobenzaprine HCl and Placebo groups. ANCOVA analysis: comparison between groups (Cyclobenzaprine HCl 5.6 mg minus Placebo). Red diamond refers to treatment differences with p<0.05, not corrected for multiple comparisons. CI refers to confidence interval.

### DETAILED DESCRIPTION

The present disclosure provides in some embodiments methods for treating or managing fibromyalgia and its associated symptoms in a subject in need thereof characterized by an early onset of one or more of: (1) a reduction in widespread pain; (2) a reduction in sleep disturbance; (3) a reduction in fatigue; (4) an improved sleep quality, or (5) an improved fibromyalgia-specific symptoms and dysfunction, comprising administering to a subject in need thereof 2.8 mg or 5.6 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight ↑-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.

The present disclosure further provides in some embodiments multiple-variable dose methods for treating or managing fibromyalgia and its associated symptoms in a subject in need thereof characterized by an early onset of one or more of: (1) a reduction in widespread pain; (2) a reduction in sleep disturbance; (3) a reduction in fatigue; (4) an improved sleep quality, or (5) an improved fibromyalgia-specific symptoms and dysfunction, comprising the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl is in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35% ±2% by weight δ-mannitol eutectic, a mixture of a 75%±2% by weight cyclobenzaprine HCl and 25%±2% by weight β-mannitol and a 65%±2% by weight cyclobenzaprine HCl and 35% ±2% by weight δ-mannitol eutectic, and a granule comprising an outer layer of a 65%±2% by weight cyclobenzaprine HCl and 35%±2% by weight δ-mannitol eutectic and an inner layer of β-mannitol, and wherein the first dosage unit and the second dosage unit further comprise a basifying agent.

The present disclosure provides in some embodiments methods for preventing or avoiding one or more of a clinically meaningful change in mean weight, a clinically meaningful change in mean systolic blood pressure or mean diastolic blood pressure, or a decline in sexual functioning in conjunction with treating or managing fibromyalgia or its associated symptoms in a subject in need thereof, the treatment or management being characterized by the transmucosal administration of in total 5.6 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65% =2% by weight cyclobenzaprine HCl and 35%±2% by weight δ-mannitol eutectic and an inner layer of β-mannitol, wherein the one or more dosage units further comprise a basifying agent.

The present disclosure provides in some embodiments methods for preventing or avoiding one or more of a clinically meaningful change in mean weight, a clinically meaningful change in mean systolic blood pressure or mean diastolic blood pressure, or a decline in sexual functioning in conjunction with treating or managing fibromyalgia or its associated symptoms in a subject in need thereof, the treatment or management being characterized by the transmucosal administration of 2.8 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the one or more dosage units further comprise a basifying agent.

The present disclosure further provides in some embodiments a method of preventing or avoiding one or more of a clinically meaningful change in mean weight, a clinically meaningful change in mean systolic blood pressure or mean diastolic blood pressure, or a decline in sexual functioning in conjunction with treating or managing fibromyalgia or its associated symptoms in a subject in need thereof, the treatment or management being characterized by the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35% ±2% by weight δ-mannitol t eutectic, a mixture of a 75%±2% by weight cyclobenzaprine HCl and 25%±2% by weight β-mannitol and a 65%±2% by weight cyclobenzaprine HCl and 35% ±2% by weight δ-mannitol eutectic, and a granule comprising an outer layer of a 65%±2% by weight cyclobenzaprine HCl and 35%±2% by weight &-mannitol eutectic and an inner layer of β-mannitol, wherein the first dosage unit and the second dosage unit further comprise a basifying agent.

### Definitions

The term “herein” means the entire application.

Unless otherwise defined herein, scientific and technical terms used in this application shall have the meanings that are commonly understood by those of ordinary skill in the art. In case of conflict, the present specification, including definitions, will control.

It should be understood that any of the embodiments described herein, including those described under different aspects of the disclosure and different parts of the specification (including embodiments described only in the Examples) can be combined with one or more other embodiments of this disclosure, unless explicitly disclaimed or improper. Combination of embodiments are not limited to those specific combinations claimed via the multiple dependent claims.

All of the publications, patents and published patent applications referred to in this application are specifically incorporated by reference herein in their entirety. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, the term “about” refers to a value or parameter that includes (and describes) embodiments that are directed to that value or parameter per se. For example, description referring to “about X” includes description of “X.” As used herein, the term “about” permits a variation of ±10% within the range of the significant digit.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article.

Notwithstanding that the disclosed numerical ranges and parameters are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments are described in terms of a Markush group or other grouping of alternatives, the present application encompasses not only the entire group listed as a whole, but each member of the group individually and all possible subgroups of the main group, and also the main group absent one or more of the group members.

Exemplary methods and materials are described herein, although methods and materials similar or equivalent to those described herein can also be used in the practice or testing of the various aspects and embodiments. The materials, methods, and examples are illustrative only and not intended to be limiting.

In order that the disclosure may be more readily understood, certain terms are first defined. These definitions should be read in light of the remainder of the disclosure as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “treat” also referred to as “manage” and their cognates refer to a full or partial amelioration or modulation of fibromyalgia or at least one discernible symptom therein characterized by one or more of (1) a reduction in widespread pain; (2) a reduction in sleep disturbance; (3) a reduction in fatigue; or (4) an improved sleep quality.

In some embodiments, “‘treat’ or ‘manage’ at least one discernible symptom” refers to a reduction of pain characterized by about 0.3 or more difference in the LS mean change in the NRS Pain Score as compared to placebo. In some embodiments, “treat” or “manage” refers to an improvement of pain score, i.e., a reduction in pain, as an associated symptom of fibromyalgia.

In some embodiments, “‘treat’ or ‘manage’ at least one discernible symptom” refers to reduction of sleep disturbance characterized by about 2.9 or more difference in the LS Mean change in PROMIS Sleep Disturbance T-score as compared to placebo. In some embodiments, “treat” or “manage” refers to an improvement in sleep quality characterized by about 0.5 or more difference in the LS Mean change in NRS Sleep Quality Score as compared to placebo.

In some embodiments, “‘treat’ or ‘manage’ at least one discernible symptom” refers to a reduction of fatigue characterized by about 2.0 or more difference in the LS Mean change in PROMIS Fatigue T-score as compared to placebo.

In some embodiments, “treat” or “manage” refers to “much improved” or “very much improved” in the context of these associated symptoms.

As used herein, the term “preventing” or “avoiding” refers to preventing or avoiding one or more of (1) a clinically meaningful change in mean weight, (2) a clinically meaningful change in mean systolic blood pressure, (3) a clinically meaningful change in mean diastolic blood pressure, or (4) a clinically meaningful decline in sexual functioning in conjunction with treating or managing fibromyalgia or its associated symptoms in a subject in need thereof.

In the embodiments of this disclosure, the cyclobenzaprine HCl-mannitol eutectic is administered together with a basifying agent. See, e.g., WO2013/188847, incorporated herein by reference.

The “basifying agent” included in the embodiments of this disclosure is selected from a group consisting of potassium dihydrogen phosphate (monopotassium phosphate, monobasic potassium phosphate, KH₂PO₄), dipotassium hydrogen phosphate (dipotassium phosphate, dibasic potassium phosphate, K₂HPO₄), tripotassium phosphate (K₃PO₄), sodium dihydrogen phosphate (monosodium phosphate, monobasic sodium phosphate, NaH₂PO₄), disodium hydrogen phosphate (disodium phosphate, dibasic sodium phosphate, Na₂HPO₄), trisodium phosphate (Na₃PO₄), bicarbonate or carbonate salts, dipotassium citrate, tripotassium citrate, disodium citrate, trisodium citrate, borate, hydroxide, silicate, nitrate, dissolved ammonia, the conjugate bases of some organic acids (including bicarbonate), and sulfide. In some embodiments, the basifying agent is potassium dihydrogen phosphate (monopotassium phosphate, monobasic potassium phosphate, KH₂PO₄) or dipotassium hydrogen phosphate (dipotassium phosphate, dibasic potassium phosphate, K₂HPO₄). In some embodiments, the basifying agent is an ingredient (and excipient) in a pharmaceutical composition suitable for transmucosal delivery and the basifying agent exerts its effects during the time the composition is being dispersed in the mucous material, while parts of the formulation are dissolving in the mucous material and for a period of time after the composition is dissolved in the mucous material. In some embodiments, the basifying agent is an ingredient (and excipient) in a tablet, and the basifying agent exerts its effects during the time the tablet is being dispersed in the mucous material, while parts of the formulation are dissolving in the mucous material and for a period of time after the tablet is dissolved in the mucous material. In some embodiments, the basifying agent is dipotassium hydrogen phosphate (K₂HPO₄). In some embodiments, the basifying agent is potassium dihydrogen phosphate (KH₂PO₄). In some embodiments, the basifying agent is disodium hydrogen phosphate (Na₂HPO₄). In some embodiments, the basifying agent is tripotassium citrate. In some embodiments, the basifying agent is trisodium citrate.

In some embodiments the cyclobenzaprine HCl-mannitol eutectic of this disclosure is selected from the group consisting of the one of the eutectics or granules referred to in paragraphs [0018]-[0022] above. In some embodiments of this disclosure, the cyclobenzaprine HCl is in the form of a 75%±2% by weight cyclobenzaprine HCl and 25%±2% by weight β-mannitol eutectic. See, e.g., WO2014/145156, incorporated herein by reference. In some embodiments, the term “cyclobenzaprine HCl eutectic” is used interchangeably with “cyclobenzaprine HCl-mannitol eutectic” and “the eutectic.”

As used herein, the term a “eutectic” “in the form of a eutectic,” or “in the form of a mannitol eutectic” refers to a mixture of chemical compounds or elements that has a single chemical composition that melts at a lower temperature than any other composition made up of the same ingredients. A composition comprising a eutectic is known as the eutectic composition and its melting temperature is known as the eutectic temperature. Eutectic compositions often have higher stability and/or dissolution rates than their non-eutectic counterparts. Because eutectics enhance dissolution, they can be employed to increase permeability in solid dispersions and dispersion systems.

Any suitable transmucosal route of administration may be employed for providing the subject with the dosage units of this disclosure. For example, transmucosal administration including sublingual, buccal, intranasal, palatal and the like may be employed as appropriate. In some embodiments the transmucosal administration is sublingual. In some embodiments, the dosage form is a sublingual tablet, a sublingual film, a sublingual liquid, sublingual powder, or a sublingual spray solution.

The terms “patient,” “subject,” “participant,” and “individual” are used interchangeably herein and refer to either a human or a non-human animal in need to treatment. These terms include mammals, such as humans, and primates (e.g., monkey). In some embodiments, the subject is a human.

The term “early onset” also refers to “rapid onset” or “rapid onset of action.”

The term “clinically meaningful change” refers to a difference that is recognized in the art that is clinically meaningful between a treatment group (e.g., Cyclobenzaprine HCl) and placebo group. In some embodiments, the methods of this disclosure avoid or prevent a clinically meaningful change in weight. In some embodiments, the methods of this disclosure avoid or prevent a clinically meaningful increase in weight. In some embodiments, the methods of this disclosure avoid or prevent a clinically meaningful decrease in weight. In some embodiments, the methods of this disclosure avoid or prevent a clinically meaningful change in one or more of mean systolic blood pressure or mean diastolic blood pressure. In some embodiments, the methods of this disclosure avoid or prevent a clinically meaningful change in mean systolic blood pressure. In some embodiments, the methods of this disclosure avoid or prevent a clinically meaningful change in mean diastolic blood pressure. In some embodiments, the methods of this disclosure avoid or prevent a clinically meaningful increase in one or more of mean systolic blood pressure or mean diastolic blood pressure. In some embodiments, the methods of this disclosure avoid or prevent a clinically meaningful increase in mean systolic blood pressure. In some embodiments, the methods of this disclosure avoid or prevent a clinically meaningful increase in mean diastolic blood pressure. In some embodiments, the methods of this disclosure avoid or prevent a clinically meaningful decline in sexual functioning. In some embodiments, the subject of the methods of the disclosure is characterized by a clinically meaningful improvement in sexual functioning.

### Methods for Treating or Managing

In some embodiments, the present disclosure provides a method for treating or managing fibromyalgia and its associated symptoms in a subject in need thereof characterized by an early onset of one or more of: (1) a reduction in widespread pain characterized by about 0.3 or more difference in the least squares (LS) mean change in the Numerical Rating Score (NRS) Pain Score; (2) a reduction in sleep disturbance characterized by about 2.9 or more difference in the LS Mean change in PROMIS Sleep Disturbance T-score; (3) a reduction in fatigue characterized by about 2.0 or more difference in the LS Mean change in PROMIS Fatigue T-score; or (4) an improved sleep quality characterized by about 0.5 or more difference in the LS Mean change in NRS Sleep Quality Score, each as compared to placebo, comprising administering to a subject in need thereof 5.6 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.

In some embodiments, the present disclosure provides a method for treating or managing fibromyalgia and its associated symptoms in a subject in need thereof characterized by an early onset of a reduction in widespread pain characterized by about 0.3 or more difference in the LS mean change in the NRS Pain Score as compared to placebo, comprising administering to a subject in need thereof 5.6 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight &-mannitol eutectic and an inner layer of β-mannitol, and the one or more dosage units further comprising a basifying agent.

In some embodiments, the present disclosure provides a method for treating or managing fibromyalgia and its associated symptoms in a subject in need thereof characterized by an early onset of a reduction in sleep disturbance characterized by about 2.9 or more difference in the LS Mean change in PROMIS Sleep Disturbance T-score as compared to placebo, comprising administering to a subject in need thereof 5.6 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.

In some embodiments, the present disclosure provides a method for treating or managing fibromyalgia and its associated symptoms in a subject in need thereof characterized by an early onset of a reduction in fatigue characterized by about 2.0 or more difference in the LS Mean change in PROMIS Fatigue T-score as compared to placebo, comprising administering to a subject in need thereof 5.6 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.

In some embodiments, the present disclosure provides a method for treating or managing fibromyalgia and its associated symptoms in a subject in need thereof characterized by an early onset of an improved sleep quality characterized by about 0.5 or more difference in the LS Mean change in NRS Sleep Quality Score as compared to placebo, comprising administering to a subject in need thereof 5.6 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.

In some embodiments, the present disclosure provides a method for treating or managing fibromyalgia and its associated symptoms in a subject in need thereof characterized by an early onset of an improved fibromyalgia-specific symptoms and dysfunction, comprising administering to a subject in need thereof 5.6 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and by weight 35%±2% δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.

In some embodiments, the present disclosure provides a method for treating or managing fibromyalgia and its associated symptoms in a subject in need thereof characterized by an early onset of one or more of: (1) a reduction in widespread pain characterized by about 0.3 or more difference in the least squares (LS) mean change in the Numerical Rating Scale (NRS) Pain Score; (2) a reduction in sleep disturbance characterized by about 2.9 or more difference in the LS Mean change in PROMIS Sleep Disturbance T-score; (3) a reduction in fatigue characterized by about 2.0 or more difference in the LS Mean change in PROMIS Fatigue T-score; or (4) an improved sleep quality characterized by about 0.5 or more difference in the LS Mean change in NRS Sleep Quality Score, each as compared to placebo, comprising administering to a subject in need thereof 2.8 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.

In some embodiments, the present disclosure provides a method for treating or managing fibromyalgia and its associated symptoms in a subject in need thereof characterized by an early onset of a reduction in widespread pain characterized by about 0.3 or more difference in the LS mean change in the NRS Pain Score as compared to placebo, comprising administering to a subject in need thereof 2.8 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.

In some embodiments, the present disclosure provides a method for treating or managing fibromyalgia and its associated symptoms in a subject in need thereof characterized by an early onset of a reduction in sleep disturbance characterized by about 2.9 or more difference in the LS Mean change in PROMIS Sleep Disturbance T-score as compared to placebo, comprising administering to a subject in need thereof 2.8 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.

In some embodiments, the present disclosure provides a method for treating or managing fibromyalgia and its associated symptoms in a subject in need thereof characterized by an early onset of a reduction in fatigue characterized by about 2.0 or more difference in the LS Mean change in PROMIS Fatigue T-score as compared to placebo, comprising administering to a subject in need thereof 2.8 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol by weight and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.

In some embodiments, the present disclosure provides a method for treating or managing fibromyalgia and its associated symptoms in a subject in need thereof characterized by an early onset of an improved sleep quality characterized by about 0.5 or more difference in the LS Mean change in NRS Sleep Quality Score as compared to placebo, comprising administering to a subject in need thereof 2.8 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.

In some embodiments, the present disclosure provides a method for treating or managing fibromyalgia and its associated symptoms in a subject in need thereof characterized by an early onset of an improved fibromyalgia-specific symptoms and dysfunction, comprising administering to a subject in need thereof 2.8 mg cyclobenzaprine HCl once daily in one or more dosage units by transmucosal administration, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the one or more dosage units further comprising a basifying agent.

In some embodiments, the one or more dosage units comprising the cyclobenzaprine HCl eutectic and the basifying agent is one dosage unit. In some embodiments, the one or more dosage units comprising the cyclobenzaprine HCl eutectic and the basifying agent is one dosage unit, the dosage unit comprising 2.8 mg of cyclobenzaprine HCl. In some embodiments, the one or more dosage units comprising the cyclobenzaprine HCl eutectic and the basifying agent are two dosage units. In some embodiments, the one or more dosage units comprising the cyclobenzaprine HCl and the basifying agent eutectic are two dosage units, each dosage unit comprising 2.8 mg of cyclobenzaprine HCl. In some embodiments, the one or more dosage units comprising the cyclobenzaprine HCl eutectic and the basifying agent are three dosage units.

In some embodiments, the present disclosure provides a multiple-variable dose method for treating or managing fibromyalgia and its associated symptoms in a subject in need thereof characterized by an early onset of one or more of: (1) a reduction in widespread pain characterized by about 0.3 or more difference in the LS mean change in the NRS Pain Score; (2) a reduction in sleep disturbance characterized by about 2.9 or more difference in the LS Mean change in PROMIS Sleep Disturbance T-score; (3) a reduction in fatigue characterized by about 2.0 or more difference in the LS Mean change in PROMIS Fatigue T-score; or (4) an improved sleep quality characterized by about 0.5 or more difference in the LS Mean change in NRS Sleep Quality Score, each as compared to placebo, comprising the transmucosal administration of; one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl is in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight-mannitol eutectic, and a granule comprising an outer layer of a 65%±2% by weight cyclobenzaprine HCl and 35% ±2% by weight δ-mannitol eutectic and an inner layer of B-mannitol, and wherein the first dosage unit and the second dosage unit further comprise a basifying agent.

In some embodiments, the present disclosure provides a multiple-variable dose method for treating or managing fibromyalgia and its associated symptoms in a subject in need thereof characterized by an early onset of a reduction in widespread pain characterized by about 0.3 or more difference in the LS mean change in the NRS Pain Score as compared to placebo, comprising the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl is in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and wherein the first dosage unit and the second dosage unit further comprise a basifying agent.

In some embodiments, the present disclosure provides a multiple-variable dose method for treating or managing fibromyalgia and its associated symptoms in a subject in need thereof characterized by an early onset of a reduction in sleep disturbance characterized by about 2.9 or more difference in the LS Mean change in PROMIS Sleep Disturbance T-score as compared to placebo, comprising the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl is in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35% ±2% by weight δ-mannitol eutectic and an inner layer of β-mannitol, and wherein the first dosage unit and the second dosage unit further comprise a basifying agent.

In some embodiments, the present disclosure provides a multiple-variable dose method for treating or managing fibromyalgia and its associated symptoms in a subject in need thereof characterized by an early onset of a reduction in fatigue characterized by about 2.0 or more difference in the LS Mean change in PROMIS Fatigue T-score as compared to placebo, comprising the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl is in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and wherein the first dosage unit and the second dosage unit further comprise a basifying agent.

In some embodiments, the present disclosure provides a multiple-variable dose method for treating or managing fibromyalgia and its associated symptoms in a subject in need thereof characterized by an early onset of an improved sleep quality characterized by about 0.5 or more difference in the LS Mean change in NRS Sleep Quality Score as compared to placebo, comprising the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl is in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the first dosage unit and the second dosage unit further comprise a basifying agent.

In some embodiments, the present disclosure provides a multiple-variable dose method for treating or managing fibromyalgia and its associated symptoms in a subject in need thereof characterized by an early onset of an improved fibromyalgia-specific symptoms and dysfunction, comprising the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl is in the form of a mannitol eutectic elected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, and the first dosage unit and the second dosage unit further comprise a basifying agent.

In some embodiments, cyclobenzaprine HCl is in the form of a eutectic comprising 75%±2% by weight cyclobenzaprine HCl and 25%±2% by weight β-mannitol eutectic. In some embodiments, cyclobenzaprine HCl is in the form of a eutectic comprising 65%±2% by weight cyclobenzaprine HCl and 35%±2% by weight δ-mannitol eutectic. In some embodiments, cyclobenzaprine HCl is in the form of a eutectic comprising a mixture of a 75%±2% by weight cyclobenzaprine HCl and 25%±2% by weight β-mannitol and a 65%±2% by weight cyclobenzaprine HCl and 35%±2% by weight δ-mannitol eutectic. In some embodiments, cyclobenzaprine HCl is in the form of a eutectic comprising a granule comprising an outer layer of a 65%±2% by weight cyclobenzaprine HCl and 35%±2% by weight δ-mannitol eutectic and an inner layer of β-mannitol. In some embodiments, the cyclobenzaprine HCl eutectic comprises 75%±2% by weight cyclobenzaprine HCl and 25%±2% by weight mannitol. In some embodiments, the eutectic that characterizes a first dosage unit and second dosage unit comprises 75%±2% by weight cyclobenzaprine HCl and 25%±2% mannitol.

In some embodiments, the one or more dosage units comprising the cyclobenzaprine HCl eutectic and basifying agent are administered at bedtime. In some embodiments, the one or more dosage units comprising the cyclobenzaprine HCl eutectic and basifying agent are administered once daily at bedtime.

In some embodiments, the transmucosal administration comprises sublingual, buccal, intranasal or palatal administration. In some embodiments, the transmucosal administration is sublingual administration. In some embodiments, the transmucosal administration is buccal administration. In some embodiments, the transmucosal administration is intranasal administration. In some embodiments, the transmucosal administration is palatal administration.

In some embodiments, the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate, trisodium citrate, borate, hydroxide, silicate, nitrate, dissolved ammonia, bicarbonate, and sulfide. In some embodiments, the basifying agent is potassium dihydrogen phosphate. In some embodiments, the basifying agent is dipotassium hydrogen phosphate. In some embodiments, the basifying agent is tripotassium phosphate. In some embodiments, the basifying agent is sodium carbonate. In some embodiments, the basifying agent is sodium bicarbonate. In some embodiments, the basifying agent is calcium carbonate. In some embodiments, the basifying agent is calcium bicarbonate. In some embodiments, the basifying agent is TRIS buffer. In some embodiments, the basifying agent is sodium dihydrogen phosphate. In some embodiments, the basifying agent is disodium hydrogen phosphate. In some embodiments, the basifying agent is trisodium phosphate. In some embodiments, the basifying agent is potassium carbonate. In some embodiments, the basifying agent is potassium bicarbonate. In some embodiments, the basifying agent is potassium acetate. In some embodiments, the basifying agent is sodium acetate. In some embodiments, the basifying agent is dipotassium citrate. In some embodiments, the basifying agent is tripotassium citrate. In some embodiments, the basifying agent is disodium citrate. In some embodiments, the basifying agent is trisodium citrate. In some embodiments, the basifying agent is borate. In some embodiments, the basifying agent is hydroxide. In some embodiments, the basifying agent is silicate. In some embodiments, the basifying agent is nitrate. In some embodiments, the basifying agent is dissolved ammonia. In some embodiments, the basifying agent is bicarbonate. In some embodiments, the basifying agent is sulfide.

In some embodiments, the pain is measured by a daily diary pain severity score using a numerical rating scale. In some embodiments, the sleep quality is measured by a daily diary score change using a numerical rating scale.

In some embodiments, early onset of the reduction in widespread pain begins about 1-4 weeks after administration of cyclobenzaprine HCl. In some embodiments, early onset of the reduction in widespread pain begins about 1-3 weeks after administration of cyclobenzaprine HCl. In some embodiments, early onset of the reduction in widespread pain begins about 1-2 weeks after administration of cyclobenzaprine HCl. In some embodiments, early onset of the reduction in widespread pain begins about 2-3 weeks after administration of cyclobenzaprine HCl. In some embodiments, early onset of the reduction in widespread pain begins about 2-4 weeks after administration of cyclobenzaprine HCL. In some embodiments, early onset of the reduction in widespread pain begins about 3-4 weeks after administration of cyclobenzaprine HCl. In some embodiments, early onset of the reduction in widespread pain begins about 1 week after administration of cyclobenzaprine HCl. In some embodiments, early onset of the reduction in widespread pain begins about 2 weeks after administration of cyclobenzaprine HCl. In some embodiments, early onset of the reduction in widespread pain begins about 3 weeks after administration of cyclobenzaprine HCl. In some embodiments, early onset of the reduction in widespread pain begins about 4 weeks after administration of cyclobenzaprine HCl.

In some embodiments, early onset of the reduction in widespread pain begins from 1-4 weeks after administration of cyclobenzaprine HCL. In some embodiments, early onset of the reduction in widespread pain begins from 1-3 weeks after administration of cyclobenzaprine HCl. In some embodiments, early onset of the reduction in widespread pain begins from 1-2 weeks after administration of cyclobenzaprine HCl. In some embodiments, early onset of the reduction in widespread pain begins from 2-3 weeks after administration of cyclobenzaprine HCl. In some embodiments, early onset of the reduction in widespread pain begins from 2-4 weeks after administration of cyclobenzaprine HCl. In some embodiments, early onset of the reduction in widespread pain begins from 3-4 weeks after administration of cyclobenzaprine HCl. In some embodiments, early onset of the reduction in widespread pain begins 1 week after administration of cyclobenzaprine HCl. In some embodiments, early onset of the reduction in widespread pain begins 2 weeks after administration of cyclobenzaprine HCl. In some embodiments, early onset of the reduction in widespread pain begins 3 weeks after administration of cyclobenzaprine HCl. In some embodiments, early onset of the reduction in widespread pain begins 4 weeks after administration of cyclobenzaprine HCL.

In some embodiments, the early onset of the reduction in sleep disturbance begins about 1-6 weeks after administration of cyclobenzaprine HCl. In some embodiments, the early onset of the reduction in sleep disturbance begins about 1-5 weeks after administration of cyclobenzaprine HCl. In some embodiments, the early onset of the reduction in sleep disturbance begins about 1-4 weeks after administration of cyclobenzaprine HCl. In some embodiments, the early onset of the reduction in sleep disturbance begins about 1-3 weeks after administration of cyclobenzaprine HCl. In some embodiments, the early onset of the reduction in sleep disturbance begins about 1-2 weeks after administration of cyclobenzaprine HCl. In some embodiments, the early onset of the reduction in sleep disturbance begins about 2-3 weeks after administration of cyclobenzaprine HCl. In some embodiments, the early onset of the reduction in sleep disturbance begins about 2-4 weeks after administration of cyclobenzaprine HCl. In some embodiments, the early onset of the reduction in sleep disturbance begins about 2-5 weeks after administration of cyclobenzaprine HCl. In some embodiments, the early onset of the reduction in sleep disturbance begins about 2-6 weeks after administration of cyclobenzaprine HCl. In some embodiments, the early onset of the reduction in sleep disturbance begins about 3-4 weeks after administration of cyclobenzaprine HCl. In some embodiments, the early onset of the reduction in sleep disturbance begins about 3-5 weeks after administration of cyclobenzaprine HCl. In some embodiments, the early onset of the reduction in sleep disturbance begins about 3-6 weeks after administration of cyclobenzaprine HCl. In some embodiments, the early onset of the reduction in sleep disturbance begins about 4-5 weeks after administration of cyclobenzaprine HCl. In some embodiments, the early onset of the reduction in sleep disturbance begins about 4-6 weeks after administration of cyclobenzaprine HCl. In some embodiments, the early onset of the reduction in sleep disturbance begins about 5-6 weeks after administration of cyclobenzaprine HCl. In some embodiments, the early onset of the reduction in sleep disturbance begins about 1 week after administration of cyclobenzaprine HCl. In some embodiments, the early onset of the reduction in sleep disturbance begins about 2 weeks after administration of cyclobenzaprine HCl. In some embodiments, the early onset of the reduction in sleep disturbance begins about 3 weeks after administration of cyclobenzaprine HCl. In some embodiments, the early onset of the reduction in sleep disturbance begins about 4 weeks after administration of cyclobenzaprine HCl. In some embodiments, the early onset of the reduction in sleep disturbance begins about 5 weeks after administration of cyclobenzaprine HCl. In some embodiments, the early onset of the reduction in sleep disturbance begins about 6 weeks after administration of cyclobenzaprine HCl.

In some embodiments, the early onset of the reduction in sleep disturbance begins from 1-6 weeks after administration of cyclobenzaprine HCl. In some embodiments, the early onset of the reduction in sleep disturbance begins from 1-5 weeks after administration of cyclobenzaprine HCl. In some embodiments, the early onset of the reduction in sleep disturbance begins from 1-4 weeks after administration of cyclobenzaprine HCl. In some embodiments, the early onset of the reduction in sleep disturbance begins from 1-3 weeks after administration of cyclobenzaprine HCl. In some embodiments, the early onset of the reduction in sleep disturbance begins from 1-2 weeks after administration of cyclobenzaprine HCl. In some embodiments, the early onset of the reduction in sleep disturbance begins from 2-3 weeks after administration of cyclobenzaprine HCl. In some embodiments, the early onset of the reduction in sleep disturbance begins from 2-4 weeks after administration of cyclobenzaprine HC1. In some embodiments, the early onset of the reduction in sleep disturbance begins from 2-5 weeks after administration of cyclobenzaprine HCl. In some embodiments, the early onset of the reduction in sleep disturbance begins from 2-6 weeks after administration of cyclobenzaprine HCl. In some embodiments, the early onset of the reduction in sleep disturbance begins from 3-4 weeks after administration of cyclobenzaprine HCl. In some embodiments, the early onset of the reduction in sleep disturbance begins from 3-5 weeks after administration of cyclobenzaprine HCl. In some embodiments, the early onset of the reduction in sleep disturbance begins from 3-6 weeks after administration of cyclobenzaprine HCl. In some embodiments, the early onset of the reduction in sleep disturbance begins from 4-5 weeks after administration of cyclobenzaprine HCl. In some embodiments, the early onset of the reduction in sleep disturbance begins from 4-6 weeks after administration of cyclobenzaprine HCl. In some embodiments, the early onset of the reduction in sleep disturbance begins from 5-6 weeks after administration of cyclobenzaprine HCl. In some embodiments, the early onset of the reduction in sleep disturbance begins 1 week after administration of cyclobenzaprine HCl. In some embodiments, the early onset of the reduction in sleep disturbance begins 2 weeks after administration of cyclobenzaprine HCl. In some embodiments, the early onset of the reduction in sleep disturbance begins 3 weeks after administration of cyclobenzaprine HCl. In some embodiments, the early onset of the reduction in sleep disturbance begins 4 weeks after administration of cyclobenzaprine HCl. In some embodiments, the early onset of the reduction in sleep disturbance begins 5 weeks after administration of cyclobenzaprine HCl. In some embodiments, the early onset of the reduction in sleep disturbance begins 6 weeks after administration of cyclobenzaprine HCl.

In some embodiments, the early onset of the reduction in fatigue begins about 1-6 weeks after administration of cyclobenzaprine HCL. In some embodiments, the early onset of the reduction in fatigue begins about 1-5 weeks after administration of cyclobenzaprine HCl. In some embodiments, the early onset of the reduction in fatigue begins about 1-4 weeks after administration of cyclobenzaprine HCl. In some embodiments, the early onset of the reduction in fatigue begins about 1-3 weeks after administration of cyclobenzaprine HCl. In some embodiments, the early onset of the reduction in fatigue begins about 1-2 weeks after administration of cyclobenzaprine HCl. In some embodiments, the early onset of the reduction in fatigue begins about 2-3 weeks after administration of cyclobenzaprine HCl. In some embodiments, the early onset of the reduction in fatigue begins about 2-4 weeks after administration of cyclobenzaprine HCl. In some embodiments, the early onset of the reduction in fatigue begins about 2-5 weeks after administration of cyclobenzaprine HCl. In some embodiments, the early onset of the reduction in fatigue begins about 2-6 weeks after administration of cyclobenzaprine HCl. In some embodiments, the early onset of the reduction in fatigue begins about 3-4 weeks after administration of cyclobenzaprine HCl. In some embodiments, the early onset of the reduction in fatigue begins about 3-5 weeks after administration of cyclobenzaprine HCl. In some embodiments, the early onset of the reduction in fatigue begins about 3-6 weeks after administration of cyclobenzaprine HCl. In some embodiments, the early onset of the reduction in fatigue begins about 4-5 weeks after administration of cyclobenzaprine HCl. In some embodiments, the early onset of the reduction in fatigue begins about 4-6 weeks after administration of cyclobenzaprine HCl. In some embodiments, the early onset of the reduction in fatigue begins about 5-6 weeks after administration of cyclobenzaprine HCl. In some embodiments, the early onset of the reduction in fatigue begins about 1 week after administration of cyclobenzaprine HCl. In some embodiments, the early onset of the reduction in fatigue begins about 2 weeks after administration of cyclobenzaprine HCl. In some embodiments, the early onset of the reduction in fatigue begins about 3 weeks after administration of cyclobenzaprine HCl. In some embodiments, the early onset of the reduction in fatigue begins about 4 weeks after administration of cyclobenzaprine HCl. In some embodiments, the early onset of the reduction in fatigue begins about 5 weeks after administration of cyclobenzaprine HCl. In some embodiments, the early onset of the reduction in fatigue begins about 6 weeks after administration of cyclobenzaprine HCl.

In some embodiments, the early onset of the reduction in fatigue begins 1-6 weeks after administration of cyclobenzaprine HCl. In some embodiments, the early onset of the reduction in fatigue begins 1-5 weeks after administration of cyclobenzaprine HCl. In some embodiments, the early onset of the reduction in fatigue begins 1-4 weeks after administration of cyclobenzaprine HCl. In some embodiments, the early onset of the reduction in fatigue begins 1-3 weeks after administration of cyclobenzaprine HCl. In some embodiments, the early onset of the reduction in fatigue begins 1-2 weeks after administration of cyclobenzaprine HCl. In some embodiments, the early onset of the reduction in fatigue begins 2-3 weeks after administration of cyclobenzaprine HCl. In some embodiments, the early onset of the reduction in fatigue begins 2-4 weeks after administration of cyclobenzaprine HCl. In some embodiments, the early onset of the reduction in fatigue begins 2-5 weeks after administration of cyclobenzaprine HCl. In some embodiments, the early onset of the reduction in fatigue begins 2-6 weeks after administration of cyclobenzaprine HCl. In some embodiments, the early onset of the reduction in fatigue begins 3-4 weeks after administration of cyclobenzaprine HCl. In some embodiments, the early onset of the reduction in fatigue begins 3-5 weeks after administration of cyclobenzaprine HCl. In some embodiments, the early onset of the reduction in fatigue begins 3-6 weeks after administration of cyclobenzaprine HCl. In some embodiments, the early onset of the reduction in fatigue begins 4-5 weeks after administration of cyclobenzaprine HCl. In some embodiments, the early onset of the reduction in fatigue begins 4-6 weeks after administration of cyclobenzaprine HCl. In some embodiments, the early onset of the reduction in fatigue begins 5-6 weeks after administration of cyclobenzaprine HCl. In some embodiments, the early onset of the reduction in fatigue begins 1 week after administration of cyclobenzaprine HCl. In some embodiments, the early onset of the reduction in fatigue begins 2 weeks after administration of cyclobenzaprine HCl. In some embodiments, the early onset of the reduction in fatigue begins 3 weeks after administration of cyclobenzaprine HCl. In some embodiments, the early onset of the reduction in fatigue begins 4 weeks after administration of cyclobenzaprine HCl. In some embodiments, the early onset of the reduction in fatigue begins 5 weeks after administration of cyclobenzaprine HCL. In some embodiments, the early onset of the reduction in fatigue begins 6 weeks after administration of cyclobenzaprine HCl.

In some embodiments, the sleep quality is prior night sleep quality. In some embodiments, the early onset of the improved sleep quality begins about 1-4 weeks after administration of cyclobenzaprine HCl. In some embodiments, the early onset of the improved sleep quality begins about 1-3 weeks after administration of cyclobenzaprine HCl. In some embodiments, the early onset of the improved sleep quality begins about 1-2 weeks after administration of cyclobenzaprine HCl. In some embodiments, the early onset of the improved sleep quality begins about 2-3 weeks after administration of cyclobenzaprine HCl. In some embodiments, the early onset of the improved sleep quality begins about 2-4 weeks after administration of cyclobenzaprine HCl. In some embodiments, the early onset of the improved sleep quality begins about 3-4 weeks after administration of cyclobenzaprine HCl. In some embodiments, the early onset of the improved sleep quality begins about 1 week after administration of cyclobenzaprine HCl. In some embodiments, the early onset of the improved sleep quality begins about 2 weeks after administration of cyclobenzaprine HCl. In some embodiments, the early onset of the improved sleep quality begins about 3 weeks after administration of cyclobenzaprine HCl. In some embodiments, the early onset of the improved sleep quality begins about 4 weeks after administration of cyclobenzaprine HCl.

In some embodiments, the early onset of the improved sleep quality begins 1-4 weeks after administration of cyclobenzaprine HCl. In some embodiments, the early onset of the improved sleep quality begins 1-3 weeks after administration of cyclobenzaprine HCl. In some embodiments, the early onset of the improved sleep quality begins 1-2 weeks after administration of cyclobenzaprine HCl. In some embodiments, the early onset of the improved sleep quality begins 2-3 weeks after administration of cyclobenzaprine HCl. In some embodiments, the early onset of the improved sleep quality begins 2-4 weeks after administration of cyclobenzaprine HCl. In some embodiments, the early onset of the improved sleep quality begins 3-4 weeks after administration of cyclobenzaprine HCl. In some embodiments, the early onset of the improved sleep quality begins 1 week after administration of cyclobenzaprine HCl. In some embodiments, the early onset of the improved sleep quality begins 2 weeks after administration of cyclobenzaprine HCl. In some embodiments, the early onset of the improved sleep quality begins 3 weeks after administration of cyclobenzaprine HCl. In some embodiments, the early onset of the improved sleep quality begins 4 weeks after administration of cyclobenzaprine HCl. In some embodiments, the subject is human.

### Methods for Improving the Tolerability and Side Effect Profile in the Treatment or Management of Fibromyalgia or its Associated Symptoms

In some embodiments, the present disclosure provides a method for preventing or avoiding one or more of a clinically meaningful change in mean weight, a clinically meaningful change in mean systolic blood pressure or mean diastolic blood pressure, or a decline in sexual functioning in conjunction with treating or managing fibromyalgia or its associated symptoms in a subject in need thereof, the treatment or management being characterized by the transmucosal administration of in total 5.6 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35% #2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65% =2% by weight cyclobenzaprine HCl and 35%±2% by weight δ-mannitol eutectic and an inner layer of β-mannitol, wherein the one or more dosage units further comprise a basifying agent.

In some embodiments, the present disclosure provides a method for preventing or avoiding a clinically meaningful change in mean weight in conjunction with treating or managing fibromyalgia or its associated symptoms in a subject in need thereof, the treatment or management being characterized by the transmucosal administration of in total 5.6 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the one or more dosage units further comprise a basifying agent.

In some embodiments, the present disclosure provides a method for preventing or avoiding a clinically meaningful change in mean systolic blood pressure or mean diastolic blood pressure in conjunction with treating or managing fibromyalgia or its associated symptoms in a subject in need thereof, the treatment or management being characterized by the transmucosal administration of in total 5.6 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65% =2% by weight cyclobenzaprine HCl and 35%±2% by weight δ-mannitol eutectic and an inner layer of β-mannitol, wherein the one or more dosage units further comprise a basifying agent.

In some embodiments, the present disclosure provides a method for preventing or avoiding a clinically meaningful change in mean systolic blood pressure in conjunction with treating or managing fibromyalgia or its associated symptoms in a subject in need thereof, the treatment or management being characterized by the transmucosal administration of in total 5.6 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight ß-mannitol and a 65%±2% by weight cyclobenzaprine HCl and 35%±2% by weight β-mannitol eutectic, and a granule comprising an outer layer of a 65%±2% by weight cyclobenzaprine HCl and 35%±2% by weight δ-mannitol eutectic and an inner layer of B-mannitol, wherein the one or more dosage units further comprise a basifying agent.

In some embodiments, the present disclosure provides a method for preventing or avoiding a clinically meaningful change in mean diastolic blood pressure in conjunction with treating or managing fibromyalgia or its associated symptoms in a subject in need thereof, the treatment or management being characterized by the transmucosal administration of in total 5.6 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the one or more dosage units further comprise a basifying agent.

In some embodiments, the present disclosure provides a method for preventing or avoiding a decline in sexual functioning in conjunction with treating or managing fibromyalgia or its associated symptoms in a subject in need thereof, the treatment or management being characterized by the transmucosal administration of in total 5.6 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the one or more dosage units further comprise a basifying agent.

In some embodiments, the present disclosure provides a method for preventing or avoiding one or more of a clinically meaningful change in mean weight, a clinically meaningful change in mean systolic blood pressure or mean diastolic blood pressure, or a decline in sexual functioning in conjunction with treating or managing fibromyalgia or its associated symptoms in a subject in need thereof, the treatment or management being characterized by the transmucosal administration of 2.8 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the one or more dosage units further comprise a basifying agent.

In some embodiments, the present disclosure provides a method for preventing or avoiding a clinically meaningful change in mean weight in conjunction with treating or managing fibromyalgia or its associated symptoms in a subject in need thereof, the treatment or management being characterized by the transmucosal administration of 2.8 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the one or more dosage units further comprise a basifying agent.

In some embodiments, the present disclosure provides a method for preventing or avoiding a clinically meaningful change in mean systolic blood pressure or mean diastolic blood pressure in conjunction with treating or managing fibromyalgia or its associated symptoms in a subject in need thereof, the treatment or management being characterized by the transmucosal administration of 2.8 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and by weight 25%±2% β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the one or more dosage units further comprise a basifying agent.

In some embodiments, the present disclosure provides a method for preventing or avoiding a clinically meaningful change in mean systolic blood pressure in conjunction with treating or managing fibromyalgia or its associated symptoms in a subject in need thereof, the treatment or management being characterized by the transmucosal administration of 2.8 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the one or more dosage units further comprise a basifying agent.

In some embodiments, the present disclosure provides a method for preventing or avoiding a clinically meaningful change in mean diastolic blood pressure in conjunction with treating or managing fibromyalgia or its associated symptoms in a subject in need thereof, the treatment or management being characterized by the transmucosal administration of 2.8 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the one or more dosage units further comprise a basifying agent.

In some embodiments, the present disclosure provides a method for preventing or avoiding a decline in sexual functioning in conjunction with treating or managing fibromyalgia or its associated symptoms in a subject in need thereof, the treatment or management being characterized by the transmucosal administration of 2.8 mg cyclobenzaprine HCl once daily in one or more dosage units to the subject, the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35% ±2% by weight δ-mannitol eutectic, a mixture of a 75%±2% by weight cyclobenzaprine HCl and 25%±2% by weight β-mannitol and a 65%±2% by weight cyclobenzaprine HCl and 35% ±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the one or more dosage units further comprise a basifying agent.

In some embodiments, cyclobenzaprine HCl is in the form of a eutectic comprising 75%±2% by weight cyclobenzaprine HCl and 25%±2% by weight β-mannitol eutectic. In some embodiments, cyclobenzaprine HCl is in the form of a eutectic comprising 65%±2% by weight cyclobenzaprine HCl and 35%±2% by weight δ-mannitol eutectic. In some embodiments, cyclobenzaprine HCl is in the form of a eutectic comprising a mixture of a 75%±2% by weight cyclobenzaprine HCl and 25%±2% by weight β-mannitol and a 65%±2% by weight cyclobenzaprine HCl and 35%±2% by weight δ-mannitol eutectic. In some embodiments, cyclobenzaprine HCl is in the form of a eutectic comprising a granule comprising an outer layer of a 65%±2% by weight cyclobenzaprine HCl and 35%±2% by weight δ-mannitol eutectic and an inner layer of β-mannitol. In some embodiments, the eutectic that characterizes a first dosage unit and a second dosage unit comprises 75%±2% by weight cyclobenzaprine HCl and 25%±2% by weight β-mannitol.

In some embodiments, the one or more dosage units comprising the cyclobenzaprine HCl-mannitol eutectic and the basifying agent are two dosage units, each dosage unit comprising 2.8 mg of cyclobenzaprine HCl. In some embodiments, the one or more dosage units comprising the cyclobenzaprine HCl-mannitol eutectic and the basifying agent is one dosage unit, the dosage unit comprising 2.8 mg of cyclobenzaprine HCl.

In some embodiments, the present disclosure provides a method for preventing or avoiding one or more of a clinically meaningful change in mean weight, a clinically meaningful change in mean systolic blood pressure or mean diastolic blood pressure, or a decline in sexual functioning in conjunction with treating or managing fibromyalgia or its associated symptoms in a subject in need thereof, the treatment or management being characterized by the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the first dosage unit and the second dosage unit further comprise a basifying agent.

In some embodiments, the present disclosure provides a method for preventing or avoiding a clinically meaningful change in mean weight in conjunction with treating or managing fibromyalgia or its associated symptoms in a subject in need thereof, the treatment or management being characterized by the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of B-mannitol, wherein the first dosage unit and the second dosage unit further comprise a basifying agent.

In some embodiments, the present disclosure provides a method for preventing or avoiding a clinically meaningful change in mean systolic blood pressure or mean diastolic blood pressure in conjunction with treating or managing fibromyalgia or its associated symptoms in a subject in need thereof, the treatment or management being characterized by the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol, wherein the first dosage unit and the second dosage unit further comprise a basifying agent.

In some embodiments, the present disclosure provides a method for preventing or avoiding a clinically meaningful change in mean systolic blood pressure in conjunction with treating or managing fibromyalgia or its associated symptoms in a subject in need thereof, the treatment or management being characterized by the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of B-mannitol, wherein the first dosage unit and the second dosage unit further comprise a basifying agent.

In some embodiments, the present disclosure provides a method for preventing or avoiding a clinically meaningful change in mean diastolic blood pressure in conjunction with treating or managing fibromyalgia or its associated symptoms in a subject in need thereof, the treatment or management being characterized by the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35% ±2% by weight δ-mannitol eutectic and an inner layer of β-mannitol, wherein the first dosage unit and the second dosage unit further comprise a basifying agent.

In some embodiments, the present disclosure provides a method for preventing or avoiding a decline in sexual functioning in conjunction with treating or managing fibromyalgia or its associated symptoms in a subject in need thereof, the treatment or management being characterized by the transmucosal administration of: one or more of a first dosage unit comprising in total 2.8 mg of cyclobenzaprine HCl to the subject once daily for about two weeks; and one or more of a subsequent second dosage unit comprising in total 5.6 mg of cyclobenzaprine HCl to the subject once daily for as long as needed, wherein the cyclobenzaprine HCl being in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35% ±2% by weight δ-mannitol eutectic and an inner layer of β-mannitol, wherein the first dosage unit and the second dosage unit further comprise a basifying agent.

In some embodiments, the cyclobenzaprine HCl-mannitol eutectic that characterizes said first dosage unit and said second dosage unit is a 75%±2% by weight cyclobenzaprine HCl and 25%±2% by weight β-mannitol eutectic.

In some embodiments, the one or more dosage units comprising the cyclobenzaprine HCl-mannitol eutectic and basifying agent are administered at bedtime. In some embodiments, the one or more of a first dosage unit comprising the cyclobenzaprine HCl-mannitol eutectic and basifying agent and a subsequent second dosage unit comprising the cyclobenzaprine HCl-mannitol eutectic and basifying agent are administered at bedtime. In some embodiments, the one or more of a first dosage unit comprising the cyclobenzaprine HCl-mannitol eutectic and basifying agent are administered at bedtime. In some embodiments, the one or more of a second dosage unit comprising the cyclobenzaprine HCl-mannitol eutectic and basifying agent are administered at bedtime.

In some embodiments, the transmucosal administration comprises sublingual, buccal, intranasal or palatal administration. In some embodiments, the transmucosal administration is sublingual administration. In some embodiments, the transmucosal administration is buccal administration. In some embodiments, the transmucosal administration is intranasal administration. In some embodiments, the transmucosal administration is palatal administration.

In some embodiments, the dosage form is a sublingual tablet, a sublingual film, a sublingual liquid, sublingual powder, or a sublingual spray solution. In some embodiments, the dosage form is a sublingual tablet. In some embodiments, the dosage form is a sublingual film. In some embodiments, the dosage form is a sublingual liquid. In some embodiments, the dosage form is sublingual powder. In some embodiments, the dosage form is a sublingual spray solution.

In some embodiments, the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate, trisodium citrate, borate, hydroxide, silicate, nitrate, dissolved ammonia, bicarbonate, and sulfide. In some embodiments, the basifying agent is potassium dihydrogen phosphate. In some embodiments, the basifying agent is dipotassium hydrogen phosphate. In some embodiments, the basifying agent is tripotassium phosphate. In some embodiments, the basifying agent is sodium carbonate. In some embodiments, the basifying agent is sodium bicarbonate. In some embodiments, the basifying agent is calcium carbonate. In some embodiments, the basifying agent is calcium bicarbonate. In some embodiments, the basifying agent is TRIS buffer. In some embodiments, the basifying agent is sodium dihydrogen phosphate. In some embodiments, the basifying agent is disodium hydrogen phosphate. In some embodiments, the basifying agent is trisodium phosphate. In some embodiments, the basifying agent is potassium carbonate. In some embodiments, the basifying agent is potassium bicarbonate. In some embodiments, the basifying agent is potassium acetate. In some embodiments, the basifying agent is sodium acetate. In some embodiments, the basifying agent is dipotassium citrate. In some embodiments, the basifying agent is tripotassium citrate. In some embodiments, the basifying agent is disodium citrate. In some embodiments, the basifying agent is trisodium citrate. In some embodiments, the basifying agent is borate. In some embodiments, the basifying agent is hydroxide. In some embodiments, the basifying agent is silicate. In some embodiments, the basifying agent is nitrate. In some embodiments, the basifying agent is dissolved ammonia. In some embodiments, the basifying agent is bicarbonate. In some embodiments, the basifying agent is sulfide.

In some embodiments, the methods of this disclosure prevent or avoid a clinically meaningful change in mean weight. In some embodiments, the clinically meaningful change in mean weight comprises a clinically meaningful increase in mean weight. In some embodiments, the clinically meaningful change in mean weight comprises a clinically meaningful decrease in mean weight.

In some embodiments, the methods of this disclosure prevent or avoid a clinically meaningful change in one or more of mean systolic blood pressure or mean diastolic blood pressure. In some embodiments, the methods of this disclosure prevent or avoid a clinically meaningful change in mean systolic blood pressure. In some embodiments, the methods of this disclosure prevent or avoid a clinically meaningful change in mean diastolic blood pressure. In some embodiments, the clinically meaningful change in one or more of the mean systolic blood pressure or the mean diastolic blood pressure comprises a clinically meaningful increase in one or more of the mean systolic blood pressure or the mean diastolic blood pressure. In some embodiments, the clinically meaningful change in the mean systolic blood pressure comprises a clinically meaningful increase in the mean systolic blood pressure. In some embodiments, the clinically meaningful change in the mean diastolic blood pressure comprises a clinically meaningful increase in the mean diastolic blood pressure.

In some embodiments, the methods of this disclosure prevent or avoid a clinically meaningful decline in sexual functioning. In some embodiments, the subject of the methods of this disclosure is characterized by improvement in sexual functioning. In some embodiments, sexual functioning comprises one or more of orgasm or completion, arousal or excitement, desire or interest, frequency of desire, or pleasure. In some embodiments, sexual functioning comprises orgasm or completion. In some embodiments, sexual functioning comprises arousal or excitement. In some embodiments, sexual functioning comprises desire or interest. In some embodiments, sexual functioning comprises frequency of desire. In some embodiments, sexual functioning comprises pleasure. In some embodiments, the subject of the methods of this disclosure characterized by a clinically meaningful improvement in orgasm or completion. In some embodiments, the subject of the methods of this disclosure is characterized by a clinically meaningful improvement in arousal or excitement. In some embodiments, the subject of the methods of this disclosure is characterized by a clinically meaningful improvement in desire or interest. In some embodiments, the subject of the methods of this disclosure is characterized by a clinically meaningful improvement in frequency of desire. In some embodiments, the subject of the methods of this disclosure is characterized by a clinically meaningful improvement in pleasure.

In some embodiments, one or more of change in mean weight, change in one or more of mean systolic blood pressure or mean diastolic blood pressure, or sexual functioning is measured after daily transmucosal administration of one or more dosage units comprising cyclobenzaprine HCl to one or more subjects in a cyclobenzaprine HCl fibromyalgia treatment group. In some embodiments, one or more of change in mean weight, change in one or more of mean systolic blood pressure or mean diastolic blood pressure, or sexual functioning is measured at 2 weeks after daily transmucosal administration of one or more dosage units comprising cyclobenzaprine HCl to one or more subjects in a cyclobenzaprine HCl fibromyalgia treatment group. In some embodiments, one or more of change in mean weight, change in one or more of mean systolic blood pressure or mean diastolic blood pressure, or sexual functioning is measured at 6 weeks after daily transmucosal administration of one or more dosage units comprising cyclobenzaprine HCl to one or more subjects in a cyclobenzaprine HCl fibromyalgia treatment group. In some embodiments, one or more of change in mean weight, change in one or more of mean systolic blood pressure or mean diastolic blood pressure, or sexual functioning is measured at 10 weeks after daily transmucosal administration of one or more dosage units comprising cyclobenzaprine HCl to one or more subjects in a cyclobenzaprine HCl fibromyalgia treatment group. In some embodiments, one or more of change in mean weight, change in one or more of mean systolic blood pressure or mean diastolic blood pressure, or sexual functioning is measured at 14 weeks after daily transmucosal administration of one or more dosage units comprising cyclobenzaprine HCl to one or more subjects in a cyclobenzaprine HCl fibromyalgia treatment group.

In some embodiments, a subject is transmucosally administered one or more dosage units of this disclosure comprising in total 5.6 mg cyclobenzaprine HCl once daily for 14 weeks. In some embodiments, a subject is transmucosally administered one or more dosage units of this disclosure comprising in total 5.6 mg cyclobenzaprine HCl once daily for up to 14 weeks. In some embodiments, a subject is transmucosally administered one or more dosage units of this disclosure comprising in total 5.6 mg cyclobenzaprine HCl once daily for 14 or more weeks. In some embodiments, a subject is transmucosally administered one or more dosage units of this disclosure comprising 2.8 mg cyclobenzaprine HCl once daily for 14 weeks. In some embodiments, a subject is transmucosally administered one or more dosage units of this disclosure comprising 2.8 mg cyclobenzaprine HCl once daily for up to 14 weeks. In some embodiments, a subject is transmucosally administered one or more dosage units of this disclosure comprising 2.8 mg cyclobenzaprine HCl once daily for 14 or more weeks.

In some embodiments, a subject is transmucosally administered one or more dosage units of this disclosure comprising in total 5.6 mg cyclobenzaprine HCl once daily for 14 weeks, wherein the one or more dosage units further comprise a basifying agent. In some embodiments, a subject is transmucosally administered one or more dosage units of this disclosure comprising in total 5.6 mg cyclobenzaprine HCl once daily for up to 14 weeks, wherein the one or more dosage units further comprise a basifying agent. In some embodiments, a subject is transmucosally administered one or more dosage units of this disclosure comprising in total 5.6 mg cyclobenzaprine HCl once daily for 14 or more weeks, wherein the one or more dosage units further comprise a basifying agent. In some embodiments, a subject is transmucosally administered one or more dosage units of this disclosure comprising 2.8 mg cyclobenzaprine HCl once daily for 14 weeks, wherein the one or more dosage units further comprise a basifying agent. In some embodiments, a subject is transmucosally administered one or more dosage units of this disclosure comprising 2.8 mg cyclobenzaprine HCl once daily for up to 14 weeks, wherein the one or more dosage units further comprise a basifying agent. In some embodiments, a subject is transmucosally administered one or more dosage units of this disclosure comprising 2.8 mg cyclobenzaprine HCl once daily for 14 or more weeks, wherein the one or more dosage units further comprise a basifying agent.

In some embodiments, a subject is transmucosally administered one or more of the second dosage unit of this disclosure comprising in total 5.6 mg cyclobenzaprine HCl once daily for up to 12 weeks. In some embodiments, a subject is transmucosally administered one or more of the second dosage unit of this disclosure comprising in total 5.6 mg cyclobenzaprine HCl once daily for 12 weeks. In some embodiments, a subject is transmucosally administered one or more of the second dosage unit comprising in total 5.6 mg cyclobenzaprine HCl once daily for 12 or more weeks.

In some embodiments, the subject is human.

### Patient Global Impression of Change (PGIC)

The Patient Global Impression of Change (PGIC) is a validated instrument used to gauge the subject's assessment of change in condition (Guy. DHEW Pub No. ADM 76-338 (1976), Dworkin et al., J Pain, 9(2):105-121 (2008)). The 7-point rating scale, which has been used by subjects to evaluate their improvement or worsening of pain, has the following options:
1=Very much improved
2=Much improved
3=Minimally improved
4=No change
5=Minimally worse
6=Much worse
7=Very much worse.

### Patient-Reported Outcome Measurement Information System (PROMIS) Scales

Patient-Reported Outcome Measurement Information System (PROMIS) is a National Institutes of Health (NIH) funded initiative to develop instruments to be used across chronic conditions (www.nihpromis.org). Three PROMIS scales include the PROMIS-Sleep disturbance scale, the PROMIS-Fatigue scale, and the PROMIS-Cognitive function scale. The scales provide questions for assessing sleep quality, severity of fatigue and cognitive function abilities, respectively, over the past 7 days using a 5-point scale ranging from 1 (not at all) to 5 (very much).

### Fibromyalgia Impact Questionnaire (FIQ)

The Fibromyalgia Impact Questionnaire (FIQ) is a 21-item self-rated instrument (0-10 numeric scale) used in the evaluation of level of function, overall impact, and symptoms due to fibromyalgia in fibromyalgia patients, which has been revised (FIQ-R) and has the same three domains as the FIQ (e.g., function, overall impact, and symptoms). See, Bennett et al., Arthritis Research & Therapy, 11:R120. The FIQ-R differs from FIQ by having modified function questions and the inclusion of questions on memory, tenderness, balance, and environmental sensitivity.

### Changes in Sexual Functioning Questionnaire (CSFQ)

The Changes in Sexual Functioning Questionnaire (CSFQ) is a 36-item clinical and research instrument identifying five scales of sexual functioning. The CSFQ has been shortened to a factor structure of a 14-item version (CSFQ-14), which yields scores for three scales corresponding to the phases of the sexual response cycle (e.g., desire, arousal, and orgasm) as well as the five scales of the original CSFQ (e.g., desire/frequency, desire/interest, arousal/excitement, orgasm/complete, and pleasure).

### EXAMPLES

### Example 1. Study Design

A randomized, double-blind, multicenter, placebo-controlled study in subjects with fibromyalgia was performed in 33 U.S. sites with a sample size of N=457 subjects with fibromyalgia as defined by 2016 Revisions to the 2010/2011 FM Diagnostic Criteria (Table 1). See, Wolfe et al., “2016 Revisions to the 2010/2011 fibromyalgia diagnostic criteria,” Semin Arthritis Rheum. 2016; 46(3):319-329. The 457 subjects were randomized in a 1:1 ratio of cyclobenzaprine HCl or placebo. All subjects received one tablet of 2.8 mg cyclobenzaprine HCl, or placebo, once daily at bedtime for the first two weeks, which was increased to two tablets of 2.8 mg cyclobenzaprine HCl (5.6 mg total), or placebo, for the remaining 12 weeks for transmucosal absorption. The cyclobenzaprine HCl was in the form of a eutectic comprising 75%±2% cyclobenzaprine HCl and 25%±2% β mannitol, and each dosage unit further comprising a basifying agent.

TABLE 1
Subject Disposition

| | | Cyclobenzaprine | |
|---|---|---|---|
| | Placebo | HCl | Total |
| Randomized | 226 | 231 | 457 |
| Completed | 179 (79.2%) | 187 (81.0%) | 366 (80.1%) |
| Discontinued | 47 (20.8%) | 44 (19.0%) | 91 (19.9%) |
| Adverse Event | 8 (3.5%) | 14 (6.1%) | 22 (4.8%) |
| Lack of Efficacy | 8 (3.5%) | 2 (0.9%) | 10 (2.2%) |
| Investigator | 2 (0.9%) | 0 (0.0%) | 2 (0.4%) |
| Decision | | | |
| Withdrew Consent | 16 (7.1%) | 14 (6.1%) | 30 (6.6%) |
| Lost to Follow Up | 10 (4.4%) | 10 (4.3%) | 20 (4.4%) |
| Pregnancy | 0 (0.0%) | 1 (0.4%) | 1 (0.2%) |
| Non-Compliance | 2 (0.9%) | 3 (1.3%) | 5 (1.1%) |
| Other | 1 (0.4%) | 0 (0.0%) | 1 (0.2%) |

The Primary Endpoint of weekly averages of daily diary average pain severity score measured was measured from baseline to Week 14 for the cyclobenzaprine HCl treatment (N=231) and the Placebo (N=225) groups. The key Secondary Endpoints (with a prespecified hierarchy used to control overall type I error) were also measured from baseline to Week 14 for the cyclobenzaprine HCl treatment (N=231) and the Placebo (N=225) groups, which include: (1) Patient Global Impression of Change responder analysis; (2) Fibromyalgia Impact Questionnaire-Revised (FIQ-R) Symptom Domain score; (3) FIQ-R Function Domain score; (4) PROMIS Sleep Disturbance instrument; (5) PROMIS Fatigue instrument; and (6) Weekly average of the daily diary assessment of sleep quality. The p-value of the Primary Endpoints and key Secondary Endpoints are listed in Table 2.

TABLE 2
Primary and Secondary Endpoints

| | Intent-to-Treat | P-Value at | Effect |
|---|---|---|---|
| Primary Endpoint | Analysis | Week 14 | size |
| Change from baseline to Week 14 | Mean Change | =0.00005 | 0.38 |
| (cyclobenzaprine HCl vs. placebo) | from Baseline | | |
| in weekly averages of daily diary | | | |
| average pain severity score | | | |
| Key Secondary Endpoints | | P-Value | |
| Patient Global Impression of | Proportion | <0.001 | |
| Change (PGIC) responder | “Much” or | | |
| analysis | “Very Much | | |
| | Improved” | | |
| Fibromyalgia Impact | Mean Change | <0.001 | |
| Questionnaire - Revised (FIQ-R) | from Baseline | | |
| Symptom Domain score | | | |
| FIQ-R Function Domain score | Mean Change | =0.001 | |
| | from Baseline | | |
| Patient-Reported Outcomes | Mean Change | <0.001 | |
| Measurement Information System | from Baseline | | |
| (PROMIS) Sleep Disturbance | | | |
| instrument | | | |
| PROMIS Fatigue instrument | Mean Change | <0.001 | |
| | from Baseline | | |
| Weekly average of the daily diary | Mean Change | <0.001 | |
| assessment of sleep quality | from Baseline | | |

### Diary Assessments

To complete a daily diary, all subjects received training that explained what is being asked of them, when to complete the diary, and how to use the diary system effectively. Subjects received instructions and a password that uniquely identifies them when they log into the diary system along with instructions about what to do if they have difficulty completing the diary.

The diary was started the evening of Visit 1 and completed daily until Visit 6 (Week 14). Subjects were instructed to complete the diary in the evening before dosing. Once the patient properly identified themselves, the diary prompted answers to the following:
(0) Rate your average pain during the past 24 hours on a scale from 0-10, where zero is no pain and 10 is worst possible pain (i.e., Numerical Rating Scale for pain).
(1) Rate your sleep quality last night on a scale from 0-10 where 0 is the best possible sleep and 10 is the worst possible sleep (i.e., Numerical Rating Scale for Sleep Quality or Prior Night Sleep Quality).

After randomization to study drug at Visit 2, the following questions were added to the daily diary (starting in the evening of the day after randomization, Day 2):
(1) Did you take your study drug last night? (Yes/No)
(2) If yes, how many tablets of study drug did you take last night? (1 tablet/2 tablets)

Subject adherence was monitored in completing the diary throughout the study, and the subject was called should any problems or significant non-adherence were observed. During the screening period, there were diary compliance requirements that must be met in order for the subject to qualify for randomization. Subjects who were randomized were encouraged to continue to complete their diary. Subjects received a reminder to complete their diary if they missed diary entries. Subjects who missed two diary entries were flagged by the IRT system, and site staff were alerted to contact subjects for re-training and to instruct the subject on the importance of routine completion of their diary.

### Primary and Secondary Outcome Measure

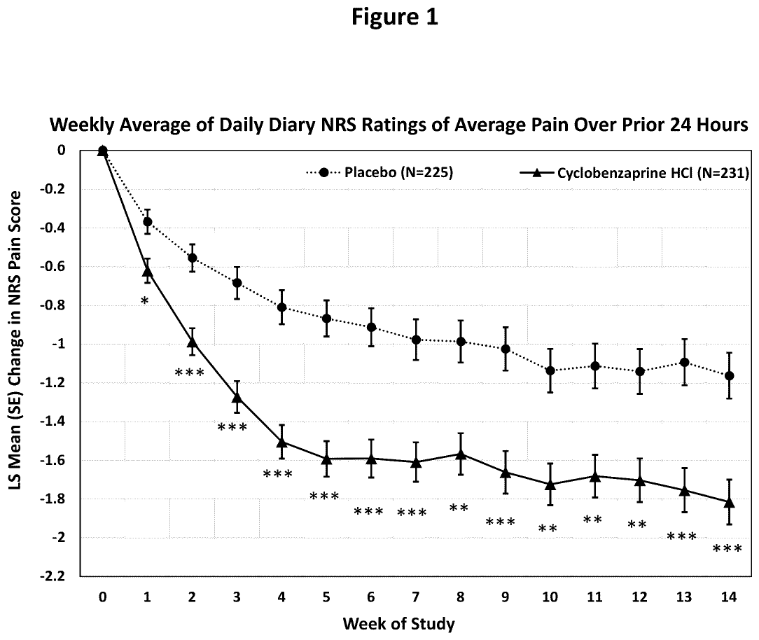

The primary outcome measure of change from baseline to Week 14 (cyclobenzaprine HCl versus placebo) in weekly averages of daily diary numerical rating scale (NRS) average pain severity score showed a reduction in widespread pain (FIG. 1). The Week 14 least squares (LS) mean (standard error (SE)) change from baseline for the cyclobenzaprine HCl was −1.82 (0.12) units and for placebo was −1.16 (0.12) units, while the least square mean difference (LSMD) from placebo was −0.65 (0.16) units; p=0.00005. The primary outcome measure was based on Mixed Model Repeated Measures with Multiple Imputation, with fixed categorical effects of treatment, center, study week, and treatment by study week interaction, as well as baseline value and baseline value-by-study week interaction.

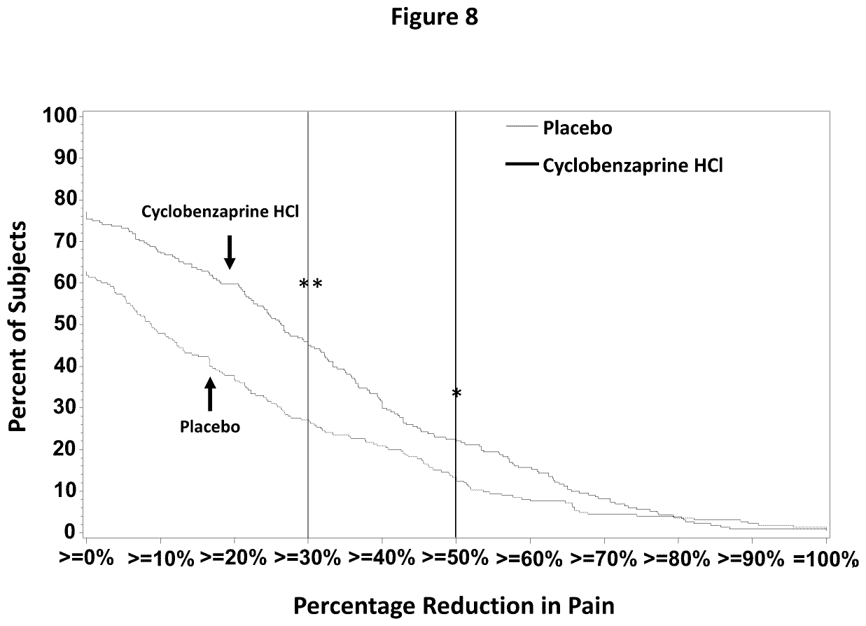

Additional analysis of the percentage of pain responders was compared between the placebo and cyclobenzaprine HCl treatment groups (FIG. 8 and Table 2). The analysis was performed using Pearson's Chi Squared test for equality of proportions.

TABLE 3
Percentage of Subjects as Pain Responders

| | 30% Reduction | 50% Reduction |
|---|---|---|
| | in Pain | in Pain |
| Placebo (% of subjects) | 27.1% | 13.3% |
| Cyclobenzaprine HCl | 45.9% | 22.5% |
| (% of subjects) | | |
| DIP (95% CI) | 18.8% | 9.2% |
| | (10.1%, 27.4%) | (2.2%, 16.2%) |
| P-value | <0.001 | =0.011 |

Abbreviations:
CI = confidence interval;
DIP = difference in proportions

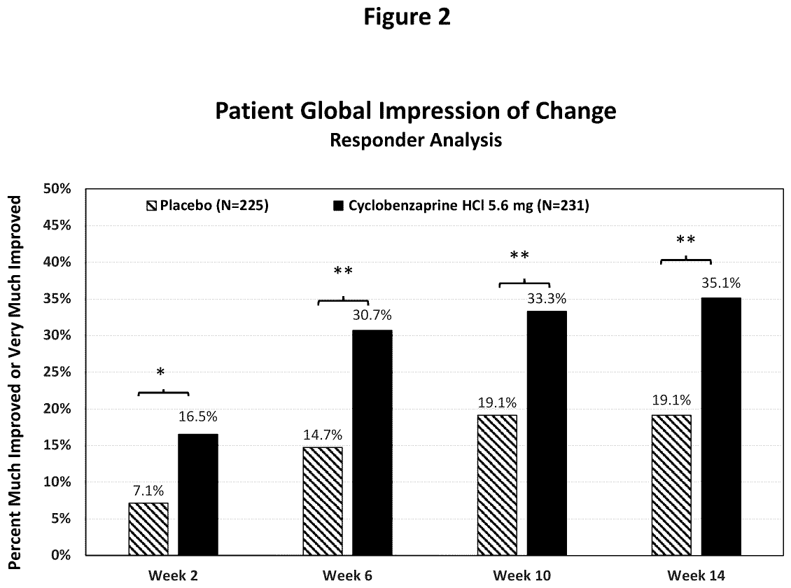

The secondary outcome measure of Patient Global Impression of Change (PGIC) Responder Analysis showed that at Week 14 cyclobenzaprine HCl responders (35.1%,) and placebo responders (19.1%) had a difference in proportions (95% CI) of 16% (7.9%, 24.0%); p=0.00013 (FIG. 2). The PGIC was based on a Pearson Chi-Squared with differences in proportions 95% CIs from difference in proportions Z-test. The Responder is defined as subject that reply ‘very much improved’ or ‘much improved’ at Week 14 and all others are non-responders.

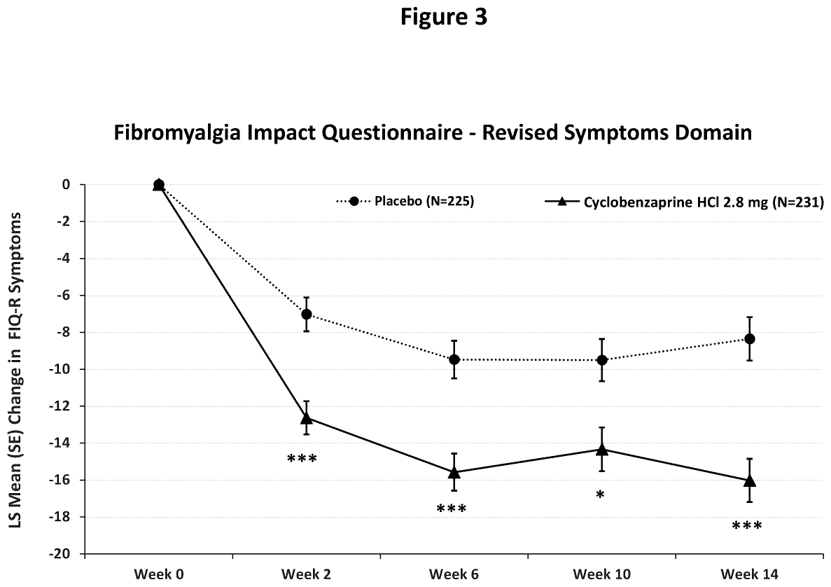

The secondary outcome measure of Week 14 LS mean (SE) change from baseline of the Fibromyalgia Impact Questionnaire-Revised (FIQ-R) Symptoms Domain for Cyclobenzaprine HCl treatment group was −16.0 (1.17) and for Placebo was −8.4 (1.17), while the LSMD (SE) from placebo was −7.7 (1.62); p=0.000002 (FIG. 3). The FIQ-R Symptoms was based on Mixed Model Repeated Measures with Multiple Imputation, with fixed categorical effects of treatment, center, study week, and treatment by study week interaction, as well as baseline value and baseline value-by-study week interaction.

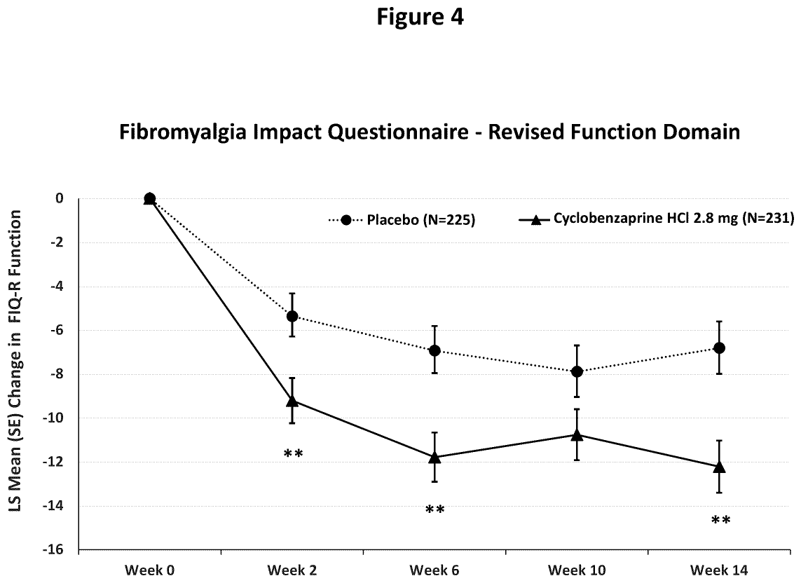

The secondary outcome measure of Week 14 LS mean (SE) change from baseline of the Fibromyalgia Impact Questionnaire-Revised (FIQ-R) Function Domain for cyclobenzaprine HCl was −12.2 (1.19) and for placebo −6.8 (1.21); LSMD (SE) from placebo −5.4 (1.66); p=0.001 (FIG. 4). The FIQ-R Function was based on Mixed Model Repeated Measures with Multiple Imputation, with fixed categorical effects of treatment, center, study week, and treatment by study week interaction, as well as baseline value and baseline value-by-study week interaction.

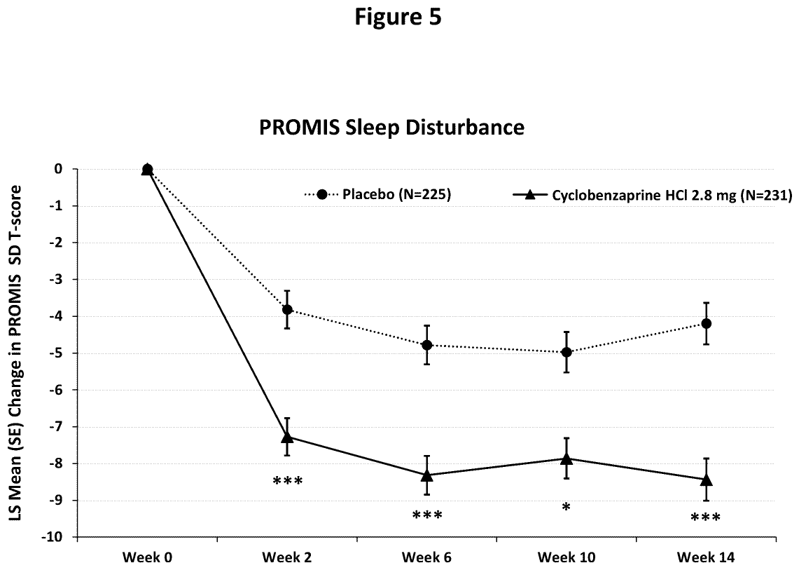

The secondary outcome measure of Week 14 LS mean (SE) change from baseline of the Patient-Reported Outcomes Measurement Information System (PROMIS) Sleep Disturbance for cyclobenzaprine HCl was −8.4 (0.57) and for placebo was −4.2 (0.56), while LSMD from placebo was −4.2 (0.79); p=0.0000001 (FIG. 5). The PROMIS Sleep Disturbance was based on Mixed Model Repeated Measures with Multiple Imputation, with fixed categorical effects of treatment, center, study week, and treatment by study week interaction, as well as baseline value and baseline value-by-study week interaction.

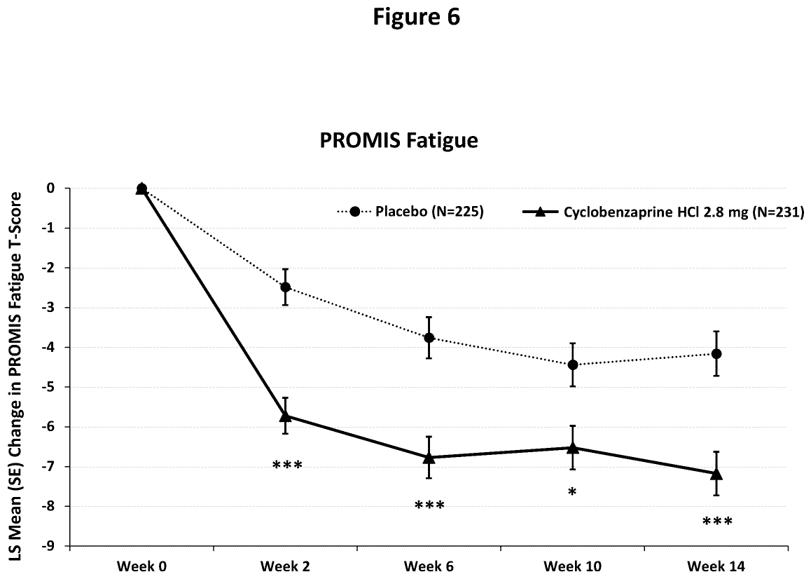

The secondary outcome measure of Week 14 LS mean (SE) change from baseline of the PROMIS Fatigue for cyclobenzaprine HCl was −7.2 (0.55) and for placebo was −4.2 (0.56), while LSMD from placebo was −3.0 (0.77); p=0.00009 (FIG. 6). The PROMIS Fatigue was based on Mixed Model Repeated Measures with Multiple Imputation, with fixed categorical effects of treatment, center, study week, and treatment by study week interaction, as well as baseline value and baseline value-by-study week interaction.

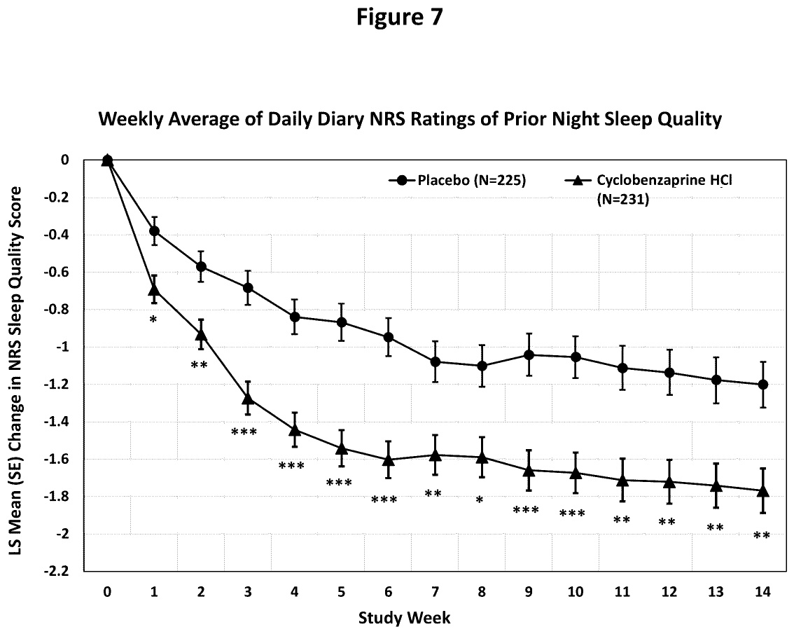

The secondary outcome measure of Week 14 LS mean (SE) change from baseline of the Weekly Average of Daily Diary NRS Ratings of Prior Night Sleep Quality for cyclobenzaprine HCl was −1.77 (0.12) units and for placebo −1.20 (0.12) units, while LSMD from placebo was −0.57 (0.17) units; p=0.0007 (FIG. 7). The NRS Ratings of Prior Night Sleep Quality was based on Mixed Model Repeated Measures with Multiple Imputation, with fixed categorical effects of treatment, center, study week, and treatment by study week interaction, as well as baseline value and baseline value-by-study week interaction.

### Adverse Events

The subjects randomized to cyclobenzaprine HCl and to placebo, 81.0% and 79.6%, respectively, completed the study. Cyclobenzaprine HCl treatment was generally well tolerated based on the adverse event (AE) profile and no new safety signals were observed. The AE-related study discontinuations occurred in 6.1% and 3.6% of subjects in the cyclobenzaprine HCl and placebo groups, respectively. The events rated as mild or moderate made up 97.2% of AEs for the placebo group and 99.1% for the cyclobenzaprine HCl group. Oral administration site AEs were higher in cyclobenzaprine HCl group (42.9%) than placebo group (10.2%). The most common oral AEs were oral hypoaesthesia, product taste abnormal, oral paraesthesia, and tongue discomfort (Table 4). Nearly all of these common oral AEs were temporally related to dosing and lasted less than 60 minutes.

TABLE 4
Safety Summary

| System Organ Class | Cyclobenzaprine HCl | Placebo | Total* |
|---|---|---|---|
| Preferred Term | (N = 231) | (N = 226) | (N = 457) |
| Oral Cavity | | | |
| Adverse Events | | | |
| Hypoaesthesia oral | 55 (23.8%) | 1 (0.4%) | 56 (12.3%) |
| Product taste abnormal | 27 (11.7%) | 2 (0.9%) | 29 (6.3%) |
| Paraesthesia oral | 16 (6.9%) | 2 (0.9%) | 18 (3.9%) |
| Tongue discomfort | 16 (6.9%) | 0 (0.0%) | 16 (3.5%) |
| Systemic Adverse | | | |
| Events | | | |
| COVID-19 | 10 (4.3%) | 7 (3.1%) | 17 (3.7%) |
| Somnolence | 7 (3.0%) | 3 (1.3%) | 10 (2.2%) |
| Headache | 7 (3.0%) | 4 (1.8%) | 11 (2.4%) |

### Serious Adverse Events (SAEs)

Three placebo subjects experienced a Serious Adverse Event (SAE), e.g., (1) Pneumonia, (2) Muscular weakness, or (3) Hypertension/Angina/Coronary Artery Disease. Two cyclobenzaprine HCl subjects experienced an SAE, e.g., (1) Renal carcinoma deemed not related to study drug or (2) Acute pancreatitis with onset 14 days after completion of treatment phase, deemed ‘possibly related’ to study drug. The outcome was ‘Recovered/Resolved.’ One subject was non-compliant with end of treatment study visits, and the last dose before onset of SAE was not known at the time that relationship with study drug was assessed.

### Other Safety Measures

### (1) Sexual Functioning

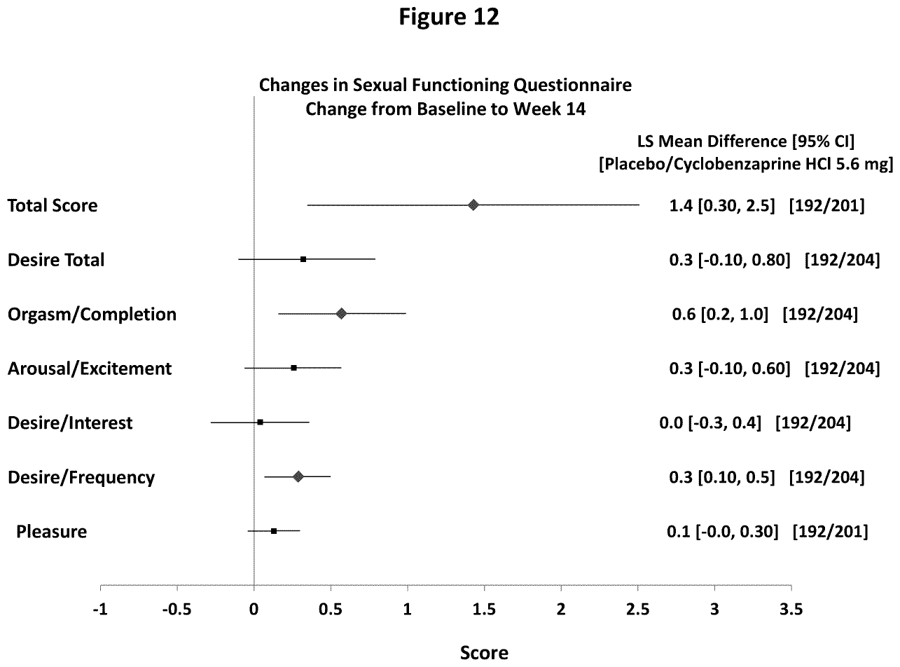

Changes in Sexual Functioning Questionnaire short form (CSFQ-14) was a safety measure in the study (FIG. 12). In females, CFSQ-14 total score improved (indicating better sexual functioning) to a greater extent in the cyclobenzaprine HCl treatment group as compared with placebo, p=0.010. Seven males in the cyclobenzaprine HCl group and 12 males in the placebo group had Week 14 CSFQ-14 completed. The desire/interest subscore improved and separated from placebo in this sample, p=0.049.

### (2) Blood Pressure

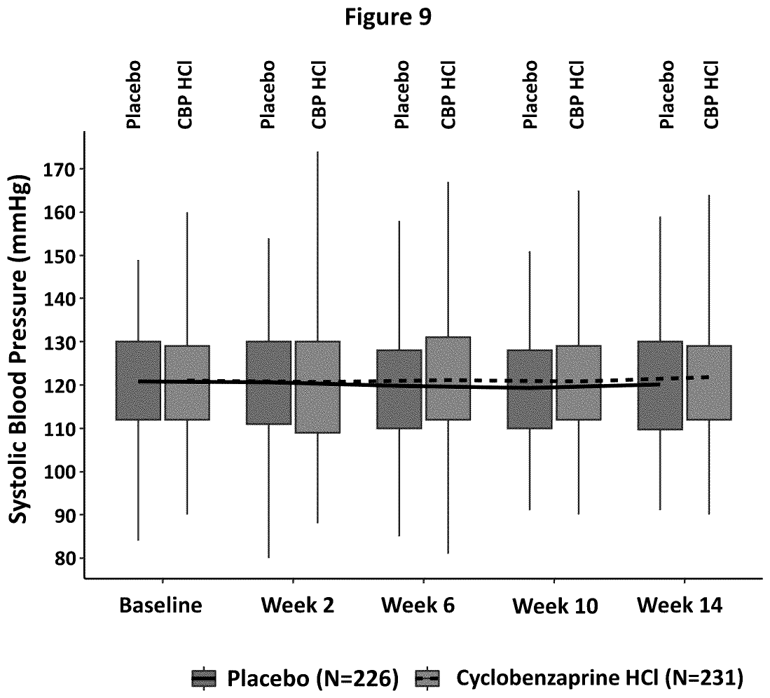

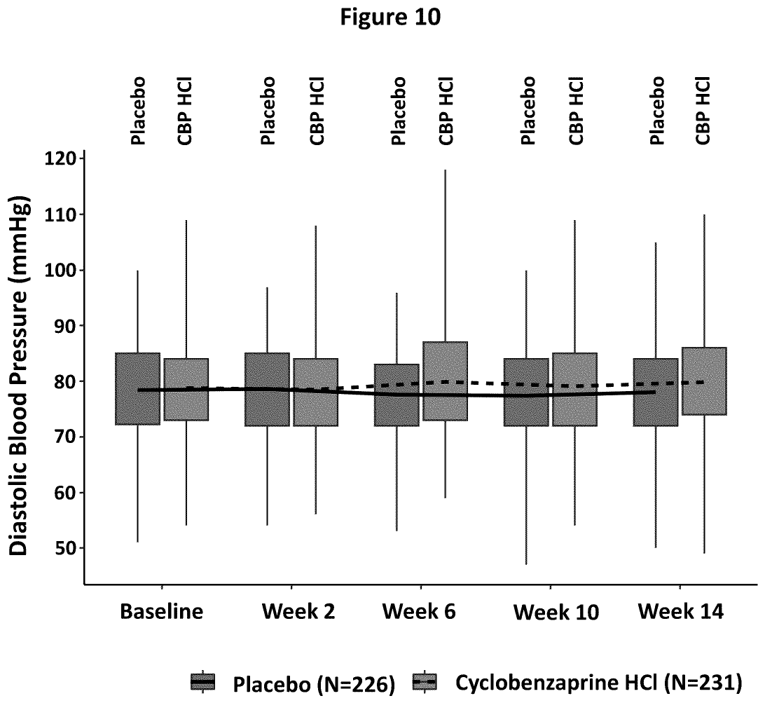

The systolic and diastolic blood pressures were measured at baseline and at Weeks 2, 6, 10, and 14 for both Cyclobenzaprine HCl and placebo groups (FIGS. 9 and 10). At Week 14, the mean (standard deviation (SD)) change of systolic blood pressure from baseline of the Cyclobenzaprine HCl group was 0.7 (12.38) mmHg and of the placebo group was 0.5 (10.42) mmHg (FIG. 9). At Week 14, the mean (SD) change of diastolic blood pressure from baseline of the Cyclobenzaprine HCl group was 1.1 (8.60) mmHg and of the placebo group was 0.2 (8.22) mmHg (FIG. 10). As these results demonstrate, there was no clinically meaningful difference in mean systolic and diastolic blood pressures between the Cyclobenzaprine HCl group and the placebo group.

### (3) Weight

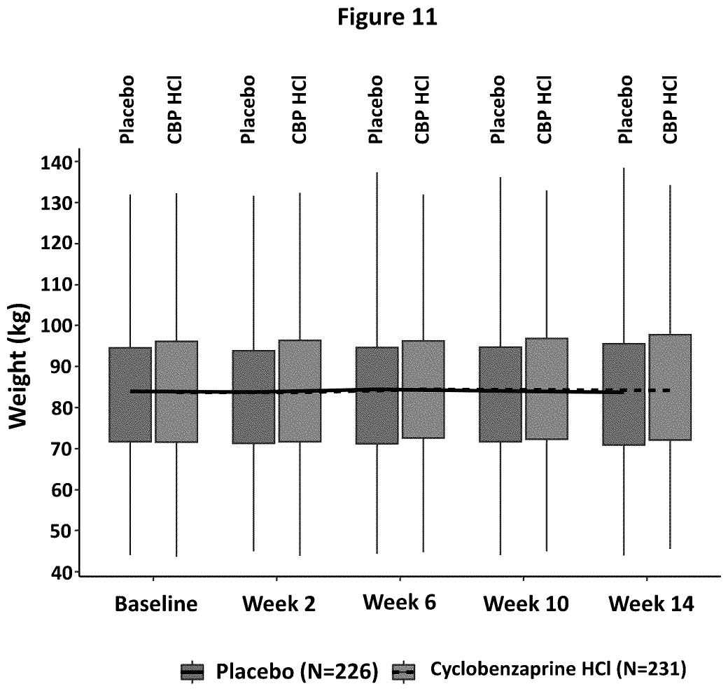

The weight was measured at baseline and at Weeks 2, 6, 10, and 14 for both Cyclobenzaprine HCl and placebo groups (FIG. 11). At Week 14, the mean (SD) change in weight from baseline of the Cyclobenzaprine HCl group was 0.02 (2.940) kg and of the placebo group was 0.20 (2.932) kg. As these results demonstrate, there was no clinically meaningful difference in mean weight between the Cyclobenzaprine HCl group and the placebo group.

1.-41. (canceled)

42. A method for treating or managing fibromyalgia and one or more of its associated symptoms in a human subject in need thereof, the method comprising:
the transmucosal administration of a total of 5.6 mg cyclobenzaprine HCl in one or more dosage units once daily to the subject, the dosage units being administered after an optional transmucosal administration of a total of 2.8 mg cyclobenzaprine HCl in one or more dosage units once daily for about two weeks,
wherein the cyclobenzaprine HCl in the one or more dosage units is in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol,
wherein each of the one or more dosage units further comprises a basifying agent; and
wherein the method is characterized by an early onset of one or more of: (1) a reduction in widespread pain characterized by an average of about 0.3 or more difference in the least squares (LS) mean change in the Numerical Rating Scale (NRS) Pain Score in a population of the subjects; (2) a reduction in sleep disturbance characterized by an average of about 2.9 or more difference in the LS Mean change in PROMIS Sleep Disturbance T-score in a population of the subjects; (3) a reduction in fatigue characterized by an average of about 2.0 or more difference in the LS Mean change in PROMIS Fatigue T-score in a population of the subjects; or (4) an improved sleep quality characterized by an average of about 0.5 or more difference in the LS Mean change in the Weekly Average of Daily Diary NRS Ratings of Prior Night Sleep Quality Score in a population of the subjects, each as compared to a population of the subjects who are administered a placebo.

43. The method for treating or managing according to claim 42, wherein the cyclobenzaprine HCl-mannitol eutectic is a 75%±2% by weight cyclobenzaprine HCl and 25% ±2% by weight β-mannitol eutectic.

44. The method for treating or managing according to claim 42, wherein the one or more dosage units that comprise the 5.6 total dose are two units, each unit comprising 2.8 mg of cyclobenzaprine HCl.

45. The method for treating or managing according to claim 42, wherein each of the one or more dosage units are administered at bedtime.

46. The method for treating or managing according to claim 42, wherein said transmucosal administration comprises sublingual, buccal, intranasal or palatal administration.

47. The method for treating or managing according to claim 46, wherein said transmucosal administration is sublingual administration.

48. The method for treating or managing according to claim 42, wherein the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate, trisodium citrate, borate, hydroxide, silicate, nitrate, dissolved ammonia, bicarbonate, and sulfide.

49. The method for treating or managing according to claim 48, wherein the basifying agent is dipotassium hydrogen phosphate.

50. The method for treating or managing according to claim 42, wherein the one or more dosage units comprising in total 5.6 mg cyclobenzaprine HCl are transmucosally administered to the subject once daily for:
(i) up to 14 weeks;
(ii) 14 or more weeks;
(iii) up to 12 weeks; or
(iv) 12 or more weeks.

51. A method for treating or managing fibromyalgia and one or more its associated symptoms in a human subject in need thereof, the method comprising:
the transmucosal administration of a total of 5.6 mg cyclobenzaprine HCl in one or more dosage units once daily to the subject, the dosage units being administered after an optional transmucosal administration of a total of 2.8 mg cyclobenzaprine HCl in one or more dosage units once daily for about two weeks,
wherein the cyclobenzaprine HCl in the one or more dosage units is in the form of a mannitol eutectic selected from the group consisting of a 75%±2% by weight cyclobenzaprine HCl and 25%±2% by weight β-mannitol eutectic, a 65%±2% by weight cyclobenzaprine HCl and 35%±2% by weight δ-mannitol eutectic, a mixture of a 75%±2% by weight cyclobenzaprine HCl and 25%±2% by weight β-mannitol and a 65%±2% by weight cyclobenzaprine HCl and 35%±2% by weight δ-mannitol eutectic, and a granule comprising an outer layer of a 65%±2% by weight cyclobenzaprine HCl and 35%±2% by weight δ-mannitol eutectic and an inner layer of β-mannitol,
wherein each of the one or more dosage units further comprise a basifying agent; and
wherein the method is characterized by the prevention or avoidance of one or more of a clinically meaningful change in mean weight, a clinically meaningful change in mean systolic blood pressure or mean diastolic blood pressure, or a decline in sexual functioning in a population of the subjects; and
wherein the clinically meaningful change in mean weight and the clinically meaningful change in mean systolic blood pressure or mean diastolic blood pressure are based on assessments at the start of the treatment or management and at one or more time points during the treatment or management, and wherein the decline in sexual functioning is based on assessments at the start of the treatment or management and at one or more time points during the treatment or management using a Changes in Sexual Functioning Questionnaire score, each as compared to a population of the subjects who are administered a placebo.

52. The method for treating or managing according to claim 51, wherein the cyclobenzaprine HCl-mannitol eutectic is a 75%±2% by weight cyclobenzaprine HCl and 25% ±2% by weight β-mannitol eutectic.

53. The method for treating or managing according to claim 51, wherein the one or more dosage units that comprise the 5.6 total dose are two units, each unit comprising 2.8 mg of cyclobenzaprine HCl.

54. The method for treating or managing according to claim 51, wherein each of the one or more dosage units are administered at bedtime.

55. The method for treating or managing according to claim 51, wherein said transmucosal administration comprises sublingual, buccal, intranasal or palatal administration.

56. The method for treating or managing according to claim 55, wherein said transmucosal administration is sublingual administration.

57. The method for treating or managing according to claim 51, wherein the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate, trisodium citrate, borate, hydroxide, silicate, nitrate, dissolved ammonia, bicarbonate, and sulfide.

58. The method for treating or managing according to claim 57, wherein the basifying agent is dipotassium hydrogen phosphate.

59. The method for treating or managing according to claim 51, wherein said sexual functioning comprises one or more of orgasm or completion, arousal or excitement, desire or interest, frequency of desire, or pleasure.

60. The method for treating or managing according to claim 51, wherein the one or more dosage units comprising in total 5.6 mg cyclobenzaprine HCl are transmucosally administered to the subject once daily for:
(i) up to 14 weeks;
(ii) 14 or more weeks;
(iii) up to 12 weeks; or
(iv) 12 or more weeks.
